(19) United States
(12) Patent Application Publication
(10) Pub. No.: US 2004/0162235 A1
(43) Pub. Date: Aug. 19, 2004

(54) Delivery of siRNA to cells using polyampholytes

(76) Inventors: Vladimir S. Trubetskoy, Madison, WI (US); David B. Rozema, Madison, WI (US); Sean D. Monahan, Madison, WI (US); Vladimir G. Budker, Middleton, WI (US); James E. Hagstrom, Middleton, WI (US); Jon A. Wolff, Madison, WI (US)

Correspondence Address: Mark K. Johnson, Mirus, 505 South Rosa Road, Madison, WI, 53719, US

(21) Appl. No.: 10/368,139

(22) Filed: Feb. 18, 2003

(51) Int. Cl. A61K 48/00

(52) U.S. Cl. 514/8; 514/44; 424/486

(57) ABSTRACT

A polyampholyte is utilized in a complex with siRNA for purposes of siRNA delivery to a cell. The complex can be formed with an appropriate amount of positive and/or negative charge such that the resulting complex can be delivered a cell in vivo or in vitro.

### FIELD OF THE INVENTION

The invention relates to compounds and methods for use in biological systems. More particularly, polyampholytes are utilized to form complexes with oligonucleotides such as siRNA for delivery to cells.

### BACKGROUND

The delivery of genetic material as a therapeutic, gene therapy, promises to be a revolutionary advance in the treatment of disease. Although, the initial motivation for gene therapy was the treatment of genetic disorders, it is becoming increasingly apparent that gene therapy will be useful for the treatment of a broad range of acquired diseases such as cancer, infectious disorders (AIDS), heart disease, arthritis, and neurodegenerative disorders (Parkinson's and Alzheimer's). Not only can functional genes be delivered to repair a genetic deficiency, but nucleic acid can also be delivered to inhibit gene expression to provide a therapeutic effect.

Inhibition of gene expression can be affected by antisense polynucleotides, siRNA mediated RNA interference and ribozymes. The transfer of nucleic acid into cells in vivo is the cardinal process of gene therapy. Transfer methods currently being explored included viral vectors and physical-chemical methods.

RNA interference (RNAi) describes the phenomenon whereby the presence of double-stranded RNA (dsRNA) of sequence that is identical or highly similar to a target gene results in the degradation of messenger RNA (mRNA) transcribed from that target gene [Sharp 2001]. It has been found that RNAi in mammalian cells is mediated by short interfering RNAs (siRNAs) of approximately 21-25 nucleotides in length [Tuschl et al. 1999 and Elbashir et al. 2001]. The ability to specifically inhibit expression of a target gene by RNAi has obvious benefits. For example, RNAi could be used to study gene function. In addition, RNAi could be used to inhibit the expression of deleterious genes and therefore alleviate symptoms of or cure disease. SiRNA delivery may also aid in drug discovery and target validation in pharmaceutical research.

A variety of methods and routes of administration have been developed to deliver pharmaceuticals that include small molecular drugs and biologically active compounds such as peptides, hormones, proteins, and enzymes to their site of action. Parenteral routes of administration include intravascular (intravenous, intra-arterial), intramuscular, intraparenchymal, intradermal, subdermal, subcutaneous, intratumor, intraperitoneal, and intralymphatic injections that use a syringe and a needle or catheter. The blood circulatory system provides systemic spread of the pharmaceutical. Polyethylene glycol and other hydrophilic polymers have provided protection of the pharmaceutical in the blood stream by preventing its interaction with blood components and to increase the circulatory time of the pharmaceutical by preventing opsonization, phagocytosis and uptake by the reticuloendothelial system. For example, the enzyme adenosine deaminase has been covalently modified with polyethylene glycol to increase the circulatory time and persistence of this enzyme in the treatment of patients with adenosine deaminase deficiency.

Transdermal routes of administration include oral, nasal, respiratory, and vaginal administration. These routes have attracted particular interest for the delivery of peptides, proteins, hormones, and cytokines, which are typically administered by parenteral routes using needles.

Liposomes have also been used as drug delivery vehicles for low molecular weight drugs and macromolecules such as amphotericin B for systemic fungal infections and candidiasis. Inclusion of anti-cancer drugs, such as adriamycin, into liposomes is being developed to increase delivery of the drugs to tumors while reducing delivery to other tissue sites thereby decreasing their toxicity. pH-sensitive polymers have been used in conjunction with liposomes for the triggered release of an encapsulated drug. For example, hydrophobically-modified N-isopropylacrylamide-methacrylic acid copolymer can render regular egg phosphatidyl choline liposomes pH-sensitive by pH-dependent interaction of grafted aliphatic chains with lipid bilayer [Meyer et al. 1998].

Non-viral vectors, such as liposomes and cationic polymers, are currently being developed to serve as gene transfer agents. Nucleic acid-containing complexes made with these vectors can be linked with proteins or other ligands for the purpose of targeting the nucleic acid to specific tissues by receptor-mediated endocytosis. It has been shown that cationic proteins like histones and protamines or synthetic polymers like polylysine, polyarginine, polyornithine, DEAE dextran, polybrene, and polyethylenimine may be effective intracellular delivery agents while small polycations like spermine are typically ineffective.

The size of a nucleic acid/polymer complex is probably critical for gene delivery in vivo. In terms of intravenous injection, nucleic acid needs to cross the endothelial barrier and reach the parenchymal cells of interest. The largest endothelia fenestrae (holes in the endothelial barrier) occur in the liver and have an average diameter of 100 nm. The trans-epithelial pores in other organs are much smaller, for example, muscle endothelium can be described as a structure which has a large number of small pores with a radius of 4 nm, and a very low number of large pores with a radius of 20-30 nm. Polycations with a charge ≧+3 facilitate nucleic acid condensation. The volume which one nucleic acid molecule occupies in a complex with polycations is drastically lower than the volume of a free nucleic acid molecule. Analysis has shown nucleic acid condensation to be favored when 90% or more of the charges along the sugar-phosphate backbone are neutralized. The size of the nucleic acid complexes is also important for the cellular uptake process. Polycations also protect nucleic acid in complexes against nuclease degradation in serum and in endosomes and lysosomes.

Optimal transfection activity in vitro and in vivo has typically required an excess of positive charge. However, the presence of an excess of polycations may be toxic to cells or may adversely affect biodistribution of the complexes in vivo. Several modifications of nucleic acid-cation particles have been created to circumvent the nonspecific interactions and toxicity of cationic particles. An example of these modifications is the attachment of steric stabilizers, e.g. polyethylene glycol, which inhibit nonspecific interactions with biological polyanions. Another example is recharging the nucleic acid particle by the addition of polyanions which interact with the cationic particle, thereby lowering its surface charge, i.e. recharging of the nucleic acid particle (U.S. Ser. No. 09/328,975). Complexes with a negative surface charge are potentially more desirable for many practical applications, such as in vivo delivery of biologically active compounds. The phenomenon of surface recharging is well known in colloid chemistry and is described in great detail for lyophobic/lyophilic systems (for example, silver halide hydrosols). Addition of polyion of opposite charge to latex particles leads to absorption of polyion on the particle surface. At the appropriate stoichiometry, the surface charge of the latex particle is thus reversed. The process is salt dependent and flocculation can occur at the neutralization point. We have demonstrated that similar layering of polyelectrolytes can be achieved on the surface of DNA/polycation particles [Trubetskoy et al. 1999]. It was shown that certain polyanions, such as poly(methacrylic acid) and poly(aspartic acid), decondensed DNA in DNA/poly-L-lysine (PLL) complexes. It was further shown that polyanions of lower charge density, such as succinylated PLL and poly(glutamic acid), did not decondense DNA in DNA/PLL (1:3) complexes even when added in 20-fold charge excess to polycation. Further studies have found that displacement effects are salt-dependent. Measurement of ζ-potential of DNA/PLL particles during titration with SPLL revealed the change of particle surface charge at approximately the charge equivalency point.

### BRIEF DESCRIPTION OF FIGURES

FIG. 1. Inhibition of firefly luciferase gene expression in mouse lungs achieved after IV administration of 50 μg siRNA (GL3) complexed with various amounts of brPEI-pAsp polyampholyte.

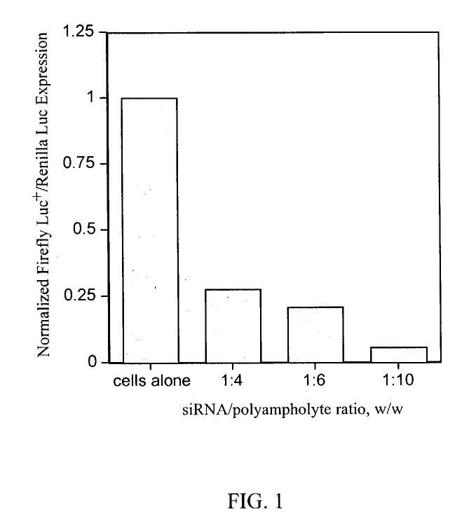

FIG. 2. Inhibition of firefly luciferase gene expression in COS7 after delivery of siRNA complexed with various amounts of brPEI-pAsp polyampholyte.

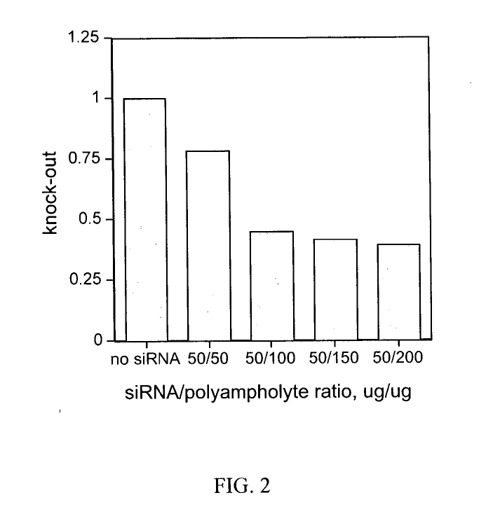

### SUMMARY

In a preferred embodiment, a process is described for delivering an siRNA to a cell, comprising: forming of a complex comprising a polyampholyte and an siRNA, and delivering the complex to a cell. Delivery of the siRNA results in inhibition of target gene expression

In a preferred embodiment, polyampholyte compounds are described that form complexes with siRNA and enhance delivery of siRNA to mammalian cells. Delivery of the siRNA results in inhibition of target gene expression.

In a preferred embodiment, we describe an in vivo process for delivery of an siRNA to a cell in a mammal for the purposes of inhibition of gene expression comprising: making an inhibitor, forming a complex comprising a polyampholyte and an siRNA, injecting the complex into the lumen of a vessel, and delivering the siRNA to the cell thereby inhibiting expression of a target gene in the cell. The complex is injected in a solution which may contain a compound or compounds which may or may not be part of the complex and aid in delivery.

In a preferred embodiment, the present invention provides a wide variety of polyampholytes with labile groups that find use in siRNA delivery systems. The labile bond may be in the main-chain of the polyampholyte, in the side chain of the polyampholyte or between the main-chain of the polyampholyte and an ionic group or other functional group. The siRNA may be linked to the polyampholyte by a labile linkage. The labile groups are selected such that they undergo a chemical transformation when present in physiological conditions. The chemical transformation may be initiated by the addition of a compound to the cell or may occur spontaneously when introduced into intra- and/or extra-cellular environments (e.g., the lower pH environment present in an endosome or in the extracellular space surrounding tumors).

In a preferred embodiment, the present invention provides siRNA delivery systems comprising: polyampholytes that contain pH-labile bonds. The systems are relatively chemically stable until they are introduced into acidic conditions. Upon delivery to an acidic environment, the labile group undergoes an acid-catalyzed chemical transformation resulting in increased delivery of the siRNA. The pH-labile bond may either be in the main-chain or in the side chain of the polyampholyte or it may be between the main-chain and an ionic group or other functional group. The siRNA may be linked to the polyampholyte by a pH-labile linkage. If the pH-labile bond occurs in the main chain, then cleavage of the labile bond results in a decrease in polyampholyte length. If the pH-labile bond occurs in the side chain, then cleavage of the labile bond results in loss of side chain atoms from the polymer. The side chain may contain an ionic group or other functional group.

In another preferred embodiment, we describe a process for extravasation of a complex comprising: forming a complex consisting of a polyampholyte and siRNA, inserting the complex into a vessel or a mammal, and delivering the complex to an extravascular space. A preferred cell is a lung cell.

In a preferred embodiment, the polyampholyte or siRNA may be modified by attachment of a functional group. The functional group can be, but is not limited to, a targeting signal or a label or other group that facilitates delivery of the inhibitor. The group may be attached to one or more of the components prior to complex formation. Alternatively, the group(s) may be attached to the complex after formation of the complex.

In a preferred embodiment the described complexes for delivery of siRNA to a cell can be used wherein the cell is located in vitro, ex vivo, in situ, or in vivo. The cell can be an animal cell that is maintained in tissue culture such as cell lines that are immortalized or transformed. The cell can be a primary or secondary cell which means that the cell has been maintained in culture for a relatively short time after being obtained from an animal. The cell can also be a mammalian cell that is within the tissue in situ or in vivo meaning that the cell has not been removed from the tissue or the animal.

In a preferred embodiment, the siRNA may be delivered to a cell in a mammal for the purposes of inhibiting a target gene to provide a therapeutic effect. The target gene is selected from the group that comprises: dysfunctional endogenous genes and viral or other infectious agent genes. Deleterious endogenous genes include dominant genes which cause disease and cancer genes.

The following description provides exemplary embodiments of the systems, compositions, and methods of the present invention. These embodiments include a variety of systems that have been demonstrated as effective delivery systems both in vitro and in vivo.

### DETAILED DESCRIPTION

The present invention relates to the compositions and methods for delivery of siRNA into cells using polyampholytes. It has previously been demonstrated that binding of negatively-charged serum components to positively charged DNA-containing complexes can significantly decrease gene transfer efficacy in vivo [Vitiello et al 1998, Ross and Hui 1999]. We found that addition of polyanions to the point of near charge reversal of the complex dramatically increases the efficacy of gene transfer mediated by DNA/polycation complexes upon IV administration in mice (U.S. patent application Ser. No. 09/328,975). We confirmed the same phenomenon for cationic lipids (PCT filing PCT/US00/22832). This improvement likely results from a protecting effect of polyanion that decreases the charge of the complex, thereby inhibiting interactions with negatively charged serum components. We have further shown that gene transfer observed with DNA/polyampholyte complexes may be based on the same phenomenon (U.S. Pat. No. 6,383,811). We now show that polyampholytes can be used to form complexes with short oligonucleotides, such as siRNA, and that these complexes can be used to delivery siRNA to mammalian cells in vivo. Delivery is increased and toxicity is reduced relative to complexes formed between polycations and siRNA. The delivered siRNA is effective in inhibiting specific gene expression in cells.

The present invention provides for the transfer of siRNA into cells in culture in vitro and to cells within tissues in situ and in vivo. For in situ and in vivo delivery, the siRNA polyampholyte complexes may be delivered intravascularly, intra-arterially, intravenously, orally, intraduodenaly, via the jejunum (or ileum or colon), rectally, transdermally, subcutaneously, intramuscularly, intraperitoneally, intraparenterally, via direct injections into tissues such as the liver, lung, heart, muscle, spleen, pancreas, brain (including intraventricular), spinal cord, ganglion, lymph nodes, lymphatic system, adipose tissues, thryoid tissue, adrenal glands, kidneys, prostate, blood cells, bone marrow cells, cancer cells, tumors, eye retina, via the bile duct, or via mucosal membranes such as in the mouth, nose, throat, vagina or rectum or into ducts of the salivary or other exocrine glands. Compounds or kits for the transfection of cells in culture are commonly sold as transfection reagents or transfection kits. Compounds for the transfection of cells in vivo in a whole organism can be sold as in vivo transfection reagents or in vivo transfection kits or as a pharmaceutical for gene therapy.

Polyampholytes are copolyelectrolytes containing both polycations and polyanions in the same polymer. In aqueous solutions polyampholytes are known to precipitate near the isoelectric point, when positive and negative charges are balanced. With an excess of either charge, polyampholytes tend to form micelle-like structures (globules). Such globules maintain tendency to bind other charged macromolecules and particles [Netz and Joanny 1998].

Conceptually, there are several ways in which one may form polyampholytes: monovalent block polyampholytes, multivalent block polyampholytes, alternating copolyampholytes and random copolyampholytes. All of these ways of constructing polyampholytes are equivalent in that they result in the formation of a polyampholyte.

Monovalent block polyampholytes are polyampholytes in which one covalent bond connects a polycation to a polyanion. Cleavage of this bond results in the formation of a polycation and a polyanion. For each polyelectrolyte there may be more than one attached polyelectrolyte of opposite charge, but the attachment between polymers is through one covalent bond.

Multivalent block polyampholytes are polyampholytes in which more than one bond connects polycation to polyanion. Cleavage of these bonds results in a polycation and a polyanion. A name for the process of connecting preformed polycations and polyanions into a multivalent block polyampholyte is crosslinking.

Alternating copolyampholytes are polyampholytes in which the cationic and anionic monomers repeat in an alternating sequence. The monomers in these polyampholytes may, but need not be, polymers themselves. Cleavage of the bonds between monomers results in anions and cations or polyanions and polycations (if the monomers are polycations and polycations).

Random copolyampholytes are polyampholytes in which the cationic and anionic monomers repeat in a random fashion. The monomers in these polyampholytes may, but need not be, polymers themselves. Cleavage of the bonds between monomers results in anions and cations or polyanions and polycations (if the monomers are polycations and polycations).

Polyampholytes may have an excess of one charge or another. For example, a polyampholyte may contain more anionic groups than cationic groups. Such a polyampholyte is termed an anionic polyampholyte. In the same way, a cationic polyampholyte contains more cationic groups than anionic groups. If a polyampholyte is composed of groups whose charge is dependent upon protonation/deprotonation for charge, then the charge of the polyampholyte itself is dependent on protonation/deprotonation, which is dependent on the pH of the solution.

A polyampholyte may contain function groups that are titratable or labile. A polyampholyte may also be modified to attach functional groups. The functional groups may be attached by labile linkages.

In this specification, the use of the term polyanion may refer to the anionic portion of a polyampholyte and the term polycation may refer to the cationic portion of a polyampholyte. In some cases, a block may be a natural protein or peptide used for cell targeting or other function. A polyanionic block such as poly(propylacrylic acid) can provide for pH-dependent membrane disruption [Murthy et al. 1999]

The polyampholytes can have other groups, functional groups, that increase their utility. These groups can be incorporated into monomers prior to polymer formation or attached to the polymer after its formation. Functional groups can also be attached to a polyampholyte after complex formation with siRNA.

A significant number of multivalent cations with widely different molecular structures have been shown to induce condensation nucleic acid. Multivalent cations with charge greater than +2 are able to condense nucleic acid into compact structures [Bloomfield 1996]. We now demonstrate that polyampholytes can form complexes with siRNA. If a polyampholyte contains one or more polyanion blocks which have higher charge density than siRNA, then the polyampholyte must have a net positive charge in excess of the negative charge contributed by the high-charge-density polyanion blocks in order to form a complex with siRNA. If the polyanion block(s) of a polyampholyte has a charge density that is lower than siRNA, then the polyampholyte may be net positively charged, net negatively charged, or charge neutral. After complex formation, the complex may be recharged with additional polyanion.

A polymer is a molecule built up by repetitive bonding together of two or more smaller units called monomers. The monomers can themselves be polymers. Polymers having fewer than 80 monomers are sometimes called oligomers. The polymer can be a homopolymer in which a single monomer is used or a copolymer in which two or more monomers are used. The polymer can be linear, branched network, star, comb, or ladder types of polymer. Types of copolymers include alternating, random, block and graft.

The main chain of a polymer is composed of the atoms whose bonds are required for propagation of polymer length. For example, in poly-L-lysine, the carbonyl carbon, α-carbon, and α-amine groups are required for the length of the polymer and are therefore main chain atoms. A side chain of a polymer is composed of the atoms whose bonds are not required for propagation of polymer length. For example in poly-L-lysine, the β, γ, δ, and ε-carbons, and ε-nitrogen are not required for the propagation of the polymer and are therefore side chain atoms.

To those skilled in the art of polymerization, there are several categories of polymerization processes that can be utilized in the described process. The polymerization can be chain or step. This classification description is more often used than the previous terminology of addition and condensation polymerization. “Most step-reaction polymerizations are condensation processes and most chain-reaction polymerizations are addition processes” [Stevens 1990]. Template polymerization can be used to form polymers from daughter polymers.

Step Polymerization: In step polymerization, the polymerization occurs in a stepwise fashion. Polymer growth occurs by reaction between monomers, oligomers and polymers. No initiator is needed since the same reaction occurs throughout and there is no termination step so that the end groups are still reactive. The polymerization rate decreases as the functional groups are consumed.

Typically, step polymerization is done in either of two different ways. In one way, the monomer has both reactive functional groups (A and B) in the same molecule so that

A-B yields -[A-B]-

Another approach is to have two difunctional monomers.

A-A+B-B yields -[A-A-B-B]-

Generally, these reactions can involve acylation or alkylation. Acylation is defined as the introduction of an acyl group (-COR) onto a molecule. Alkylation is defined as the introduction of an alkyl group onto a molecule.

If functional group A is an amine then B can be (but is not restricted to) an isothiocyanate, isocyanate, acyl azide, N-hydroxysuccinimide, sulfonyl chloride, aldehyde (including formaldehyde and glutaraldehyde), ketone, epoxide, carbonate, imidoester, carboxylate activated with a carbodiimide, alkylphosphate, arylhalides (difluoro-dinitrobenzene), anhydride, or acid halide, p-nitrophenyl ester, o-nitrophenyl ester, pentachlorophenyl ester, pentafluorophenyl ester, carbonylimidazole, carbonyl pyridinium, or carbonyl dimethylaminopyridinium. In other terms when function A is an amine then function B can be acylating or alkylating agent or amination agent.

If functional group A is a thiol (also called a sulflhydryl) then function B can be (but is not restricted to) an iodoacetyl derivative, maleimide, aziridine derivative, acryloyl derivative, fluorobenzene derivatives, or disulfide derivative (such as a pyridyl disulfide or 5-thio-2-nitrobenzoic acid {TNB} derivatives).

If functional group A is carboxylate then function B can be (but is not restricted to) a diazoacetate or an amine in which a carbodiimide is used. Other additives may be utilized such as carbonyldiimidazole, dimethylaminopyridine (DMAP), N-hydroxysuccinimide or alcohol using carbodiimide and DMAP.

If functional group A is a hydroxyl then function B can be (but is not restricted to) an epoxide, oxirane, or an amine in which carbonyldiimidazole or N,N′-disuccinimidyl carbonate, or N-hydroxysuccinimidyl chloroformate or other chloroformates are used.

If functional group A is an aldehyde or ketone then function B can be (but is not restricted to) an hydrazine, hydrazide derivative, amine (to form a Schiff Base; an imine or iminium that may or may not be reduced by reducing agents such as NaCNBH₃) or hydroxyl compound to form a ketal or acetal.

Yet another approach is to have one difunctional monomer so that A-A plus another agent yields -[A-A]-. If function A is a thiol group then it can be converted to disulfide bonds by oxidizing agents such as iodine (12) or NaIO₄ (sodium periodate), or oxygen (O₂). Function A can also be an amine that is converted to a thiol group by reaction with 2-iminothiolate (Traut's reagent) which then undergoes oxidation and disulfide formation. Disulfide derivatives (such as a pyridyl disulfide or 5-thio-2-nitrobenzoic acid {TNB} derivatives) can also be used to catalyze disulfide bond formation.

Functional group A or B in any of the above examples could also be a photoreactive group such as aryl azide (including halogenated aryl azide), diazo, benzophenone, alkyne or diazirine derivative.

Reactions of the amine, hydroxyl, thiol, carboxylate groups yield chemical bonds that are described as amide, amidine, disulfide, ethers, esters, enamine, imine, urea, isothiourea, isourea, sulfonamide, carbamate, alkylamine bond (secondary amine), carbon-nitrogen single bonds in which the carbon contains a hydroxyl group, thioether, diol, hydrazone, diazo, or sulfone.

Chain Polymerization: In chain-reaction polymerization growth of the polymer occurs by successive addition of monomer units to limited numbers of growing chains. The initiation and propagation mechanisms are different and there is usually a chain-terminating step. The polymerization rate remains constant until the monomer is depleted.

Monomers containing vinyl, acrylate, methacrylate, acrylamide, methaacrylamide groups can undergo chain reaction which can be radical, anionic, or cationic. Chain polymerization can also be accomplished by cycle or ring opening polymerization. Several different types of free radical initiators could be used that include peroxides, hydroxy peroxides, and azo compounds such as 2,2′-Azobis(-amidinopropane)dihydrochloride (AAP).

Types of Monomers: A wide variety of monomers can be used in the polymerization processes. These include positively charged organic monomers such as amines, imidine, guanidine, imine, hydroxylamine, hydrozyine, heterocycles (like imidazole, pyridine, morpholine, pyrimidine, or pyrene. The amines could be pH-sensitive in that the pKa of the amine is within the physiologic range of 4 to 8. Specific amines include spermine, spermidine, N,N′-bis(2-aminoethyl)-1,3-propanediamine (AEPD), and 3,3′-Diamino-N,N-dimethyldipropylammonium bromide.

Monomers can also be hydrophobic, hydrophilic or amphipathic. Amphipathic compounds have both hydrophilic (water-soluble) and hydrophobic (water-insoluble) parts. Hydrophilic groups indicate in qualitative terms that the chemical moiety is water-preferring. Typically, such chemical groups are water soluble and are hydrogen bond donors or acceptors with water. Examples of hydrophilic groups include compounds with the following chemical moieties carbohydrates; polyoxyethylene, peptides, oligonucleotides and groups containing amines, amides, alkoxy amides, carboxylic acids, sulfurs, or hydroxyls. Hydrophobic groups indicate in qualitative terms that the chemical moiety is water-avoiding. Typically, such chemical groups are not water soluble, and tend not to hydrogen bond. Hydrocarbons are hydrophobic groups. Monomers can also be intercalating agents such as acridine, thiazole orange, or ethidium bromide.

The polymers can also contain cleavable groups within themselves. When attached to the targeting group, cleavage leads to reduced interaction between the complex and the receptor for the targeting group. Cleavable groups include but are not restricted to disulfide bonds, diols, diazo bonds, ester bonds, sulfone bonds, acetals, ketals, enol ethers, enol esters, enamines and imines.

A polyelectrolyte, or polyion, is a polymer possessing more than one charge, i.e. a polymer that contains groups that have either gained or lost one or more electrons. A polycation is a polyclectrolyte possessing net positive charge, for example PLL hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polyelectrolyte containing a net negative charge. The polyanion can contain monomer units that are charge negative, charge neutral, or charge positive, however, the net charge on the polymer must be negative. A polyanion can also mean a non-polymeric molecule that contains two or more negative charges. The term polyelectrolyte includes polycation, polyanion, zwitterionic polymers, and neutral polymers. The term zwitterionic refers to the product (salt) of the reaction between an acidic group and a basic group that are part of the same molecule.

A copolyelectrolyte is a polyelectrolyte that contains both negative and positive charges.

In forming polyampholytes, a wide variety of charged monomers can be used in the polymerization processes. Positively charge monomers may be selected from the group comprising: amines, amine salts, alkylamine, aryl amine, aralkylamine, imidine, guanidine, imine, hydroxylamine, hydrazine, heterocycles like imidazole, pyridine, morpholine, pyrimidine, piperazine, pyrazine, pyrene, oxazoline, oxazole, and oxazolidine. Polycations made from such monomers may be selected from the group comprising: poly-L-lysine, poly-D-lysine, poly-L,D-lysine, polyethylenimine (linear and/or branched), polyallylamine, poly-L-ornithine, poly-D-ornithine, poly-L,D-ornithine, polyvinylamine, natural cationic proteins, synthetic cationic proteins, synthetic cationic peptides and synthetic polymers. Positive charges on the monomer or in the polymer can be pH-sensitive in that the pKa of the amine is within the physiological range of 4 to 8. Specific pH-sensitive amines and polyamines include spermine, spermidine, N,N′-bis(2-aminoethyl)-1,3-propanediamine (AEPD), and 3,3′-Diamino-N,N-dimethyldipropylammonium bromide. Negatively charged monomers may be selected from the group comprising: sulfates, sulfonates, carboxylates, and phosphates, may be used in the polymerization process. Polyanions may be selected from the group comprising: nucleic acids, polysulfonylstyrene, heparin sulfate poly(acrylic acid), and poly(propylacrylic acid), poly-L-aspartic acid, poly-D-aspartic acid, poly-L,D-aspartic acid, poly-L-glutamic acid, poly-D-glutamic acid, poly-L,D-glutamic acid, succinylated poly-L-lysine, succinylated poly-D-lysine, succinylated poly-L,D-lysine, succinylated polyethyleneimine, succinylated polyallylamine, succinylated poly-L-ornithine, succinylated poly-D-ornithine, succinylated poly-L,D-ornithine, succinylated polyvinylamine, polymethacrylic acid, dextran sulfate, heparin, hyaluronic acid, natural anionic proteins, synthetic anionic proteins, and synthetic anionic peptides.

In addition to charge, monomers can also be hydrophobic, hydrophilic or amphipathic. Monomers can also be intercalating agents such as acridine, thiazole orange, or ethidium bromide. Monomers can contain chemical moieties that can be modified before or after polymerization including (but not limited to) amines (primary, secondary, and tertiary), amides, carboxylic acid, ester, hydroxyl, hydrazine, alkyl halide, aldehyde, and ketone. A pH-labile polyampholyte can contain a chelator and be a polychelator.

The present invention provides for the formation of siRNA/polyampholyte complexes in which the polyampholyte contains a labile bond(s). The labile bond may occur within the backbone of the polyampholyte, between the polymer backbone and the charged ions, or between the polyampholyte and the siRNA or other functional group, such as a membrane active compound. The labile bond is then cleaved or altered once the complex is in a particular environment. This cleavage or alteration results in increased delivery of the siRNA. Cleavage may result in an increased number of molecules in an internal organelle of a cell such as an endosome. The resultant increase in osmotic pressure within the organelle may cause swelling and rupture of the organelle and thus facilitate release into the cell cytoplasm of co-delivered siRNA. Cleavage or alteration of labile bonds can also result in increased membrane activity of the polyampholyte or functional components of the polyampholyte complex. If the polyampholyte backbone is hydrophobic, cleavage of ionic groups would permit interaction of the backbone with membrane. Cleavage may also result in the release of one or more components from the complex.

Labile bonds or linkages may be selected from the group comprising: pH sensitive bonds, labile disulfide bonds (which are cleaved by reducing agents such as glutathione), bonds cleaved by enzymatic activity, hydrolytic bonds, lactone/lactam forming bonds, photolytic bonds, chelative bonds, diols, diazo bonds, ester bonds, arylsilanes, vinylsalines, allylsilanes, ester bonds, sulfone bonds, enol ethers, imminiums, and enamines. Disulfide bonds are more readily cleaved in the cytoplasm than in the extracellular milieu because of the higher concentration of the reducing agents such as glutathione present in the cytoplasm of a cell. pH labile bonds may be selected from the group comprising: acetals, ketals, silyl ether, silazane, silicon-ozygen-carbon bonds, imine, acid esters, acid thioesters, derivatives of citriconic anhydride, derivatives of maleic anhydride, derivatives of a crown ether (or azacrown ether, or thiacrown ether). The conditions under which a labile group will undergo transformation can be controlled by altering the chemical constituents of the molecule containing the labile group. For example, addition of particular chemical moieties (e.g., electron acceptors or donors) near the labile group can effect the particular conditions under which chemical transformation will occur.

Amine-containing polycations may be converted to polyanions by reaction with cyclic anhydrides such as succinic anhydride, glutaric anhydride, and 2-propionic-3-methylmaleic anhydride (carboxy-dimethylmaleic anhydride, CDM). Examples of such polyanions include, but are not limited to, succinylated and glutarylated poly-L-lysine, succinylated and glutarylated polyallylamine and CDM-polylysine. CDM-polylysine is also an example of a pH-sensitive polyanion containing a pH labile linkage. At acidic pH, the CDM side chain group is readily cleaved, regenerating the cationic polylysine polymer.

An example of a labile block polyampholyte composed of a labile constituent polyanion is fully maleamylated PLL that has been reacted with a mixture of 2-propionic-3-methylmaleic anhydride and a thioester derivative of 2-propionic-3-methylmaleic anhydride. The thioester provides an activated ester that reacts amines of cysteine groups. Addition of this labile polyanion to a cysteine-containing polycation results in the formation of a multivalent block polyampholyte.

An example of a pH-labile bond in the side chain of a polyampholyte is partially 2,3-dimethylmaleamylated poly-L-lysine, which is a random copolyampholyte. This polyampholyte is formed by the reaction of poly-L-lysine with less than one equivalent of 2,3-dimethylmaleic anhydride or 2,3-dimethylmaleic anhydride derivative under basic conditions. The modification of the poly-L-lysine is in the side chain and conversion of the 2,3-dimethylmaleamic side chain to poly-L-lysine and 2,3-dimethylmaleic anhydride under acid conditions does not result in a cleavage of the polymer main, but in a cleavage of the side chain.

A labile bond between a labile polyanion and a polycation may be made by formation of a labile polyanion by reaction of PLL with a mixture of 2-propionic-3-methylmaleic anhydride and an aldehyde derivative of 2-propionic-3-methylmaleic anhydride. The aldehyde is able to form an imine bond with an amine. Addition of this labile polyanion to a polyamine results in the formation of a multivalent block polyampholyte in which the connection between polycation and polyanion is labile.

Functional groups which are protonated in the pH range 5-7 (the pH range in the endosome) can be incorporated into a polyampholyte. Their incorporation causes the charge of the siRNA delivery system to change as the pH changes. This “buffering” of the endosome by the delivery system causes an increase in the amount of protons needed for a drop in pH. It is postulated that this increase in the amount of protons causes a swelling and bursting of the endosome. This buffering and swelling of the endosome is one hypothesized to be the means by which polyethylenimine aids in DNA transfection.

Block polyampholytes can contain pH-titratable groups. Either constituent polymer or both polymers may contain pH-titratable groups, but covalent attachment of the polymers results in a pH-titratable polyampholyte. Examples of polycations that contain the pH-titratable groups include polymers that contain imidazole groups such as polyhistidine, copolymers of histidine and polylysine, and imidazole-modified and histidylated polyamines (polyamines that have had their side chains modified to attach imidazole groups or histidine groups). An example of these modified polyamines is the acylation of polyamines with imidazole acetic acid. Polymers MC#510 and MC#486 (see examples) are imidazole-containing polymers with net negative charge. Examples of a polyanions that contain pH-titratable groups include any polymer containing carboxylic acid groups (pKa ca 4-5) such as polyaspartic acid, polyglutamic acid, succinylated PLL, polyacrylic acid, and polymethacrylic acid.

Formation of a covalent bond (or bonds) between polycations and polyanions containing pH-titratable groups results in the formation of a polyampholyte containing pH-titratable group. If one bond is formed then it is a monovalent block polyampholyte. If more than one bond is formed then it is a multivalent block polyampholyte.

A polyampholyte may include functional groups that increase their utility. These groups can be incorporated into monomers prior to polymer formation or attached to the polymer after its formation. Functional groups may be selected from the group comprising: targeting groups, signals, ligands, nuclear targeting signals, membrane active compounds, reporter molecules, marker molecules, spacers, steric stabilizers, chelators, interaction modifiers, polycations, polyanions, and polymers.

The siRNA, polyampholyte, or siRNA/polyampholyte complex may be modified with an interaction modifier such that interactions between the siRNA, polyampholyte or complex and its environs is altered. For example, attachment of nonionic hydrophilic groups, such as polyethylene glycol and polysaccharides (e.g., starch), may decrease self-association and interactions with other molecules such as serum compounds and cellular membranes. This decrease in interactions may be necessary for transport of the siRNA to the cell. However these molecules may inhibit cellular uptake, activity of other attached functional groups, or release of siRNA. Likewise, cell targeting ligands aid in transport to a cell but may not be necessary, and may inhibit, transport into a cell. Therefore, the modification may be reversible.

Many membrane active compounds, such as the peptides melittin and pardaxin and various viral proteins and peptides, are effective in disrupting cellular membranes. They are thus potentially useful in disrupting endosomes to affect release of endosomal contents into the cytoplasm. However, because of their inherent membrane activity, these agents are toxic to cells both in vitro and in vivo. In order to decrease the non-selective toxicity of the membrane active compounds, the present invention provides techniques to complex or modify the agents in ways which reversibly block or inhibit membrane activity. The membrane active compounds may be reversibly inactivated by directly modifying reactive groups, such as amines, on the membrane active compounds. The membrane active compounds may also be inactivated by their reversible incorporation into a polyampholyte complex. Membrane activity is then restored under appropriate conditions following the chemical conversion of one or more labile bonds or protonatable groups. Using pH labile bonds, membrane active compounds may be used to assist in the disruption of endosomes or other acidic cellular compartments or to deliver siRNA to acidic tissue such as tumors. Labile bonds may also be cleaved by the delivery of a cleaving agent at a time or location when it would be most beneficial. To demonstrate this principle, we synthesized polyampholytes formed by the reversible acylation of a membrane active polycation by derivatives of maleic anhydride. For example, the peptide melittin (GIGAVLKVLTTGLPALISWIKRKRQQ; SEQ ID 1) is reversible acylated by derivatives of maleic anhydride. Upon reaction with anhydride, the melittin becomes a negatively-charged polyampholyte containing four negative charges from modified amines and two positive charges from the unreactive arginine groups. When the maleamate groups cleave under acidic conditions, the melittin becomes cationic and much more membrane disruptive. In the same way, other membrane active polycations can also be reversibly modified to become labile polyampholytes.

Definitions: To facilitate an understanding of the present invention, a number of terms and phrases are defined below:

The delivery of a biologically active compound is commonly known as “drug delivery”. A biologically active compound, such as siRNA, is delivered if it becomes associated with the cell or organism. The compound can be in the circulatory system, intravessel, extracellular, on the membrane of the cell or inside the cytoplasm, nucleus, or other organelle of the cell.

Parenteral routes of administration include intravascular (intravenous, intra-arterial), intramuscular, intraparenchymal, intradermal, subdermal, subcutaneous, intratumor, intraperitoneal, intrathecal, subdural, epidural, and intralymphatic injections that use a syringe and a needle or catheter. An intravascular route of administration enables siRNA to be delivered to cells more evenly distributed than direct injections. Intravascular herein means within a tubular structure called a vessel that is connected to a tissue or organ within the body. Within the cavity of the tubular structure, a bodily fluid flows to or from the body part. Examples of bodily fluid include blood, cerebrospinal fluid (CSF), lymphatic fluid, or bile. Examples of vessels include arteries, arterioles, capillaries, venules, sinusoids, veins, lymphatics, and bile ducts. The intravascular route includes delivery through the blood vessels such as an artery or a vein. An administration route involving the mucosal membranes is meant to include nasal, bronchial, inhalation into the lungs, or via the eyes. Other routes of administration include intraparenchymal into tissues such as muscle (intramuscular), liver, brain, and kidney. Transdermal routes of administration have been effected by patches and ionotophoresis. Other epithelial routes include oral, nasal, respiratory, and vaginal routes of administration.

Extravascular means outside of a vessel such as a blood vessel. Extravascular space means an area outside of a vessel. Space may contain biological matter such as cells and does not imply empty space. Extravasation means the escape of material such as compounds and complexes from the vessel into which it is introduced into the parenchymal tissue or body cavity.

A delivery system is the means by which a biologically active compound becomes delivered. That is all compounds, including the biologically active compound itself, that are required for delivery and all procedures required for delivery including the form (such volume and phase (solid, liquid, or gas)) and method of administration (such as but not limited to oral or subcutaneous methods of delivery).

The process of delivering a nucleic acid such as siRNA to a cell has been commonly termed transfection or the process of transfecting and also it has been termed transformation. The term transfecting as used herein refers to the introduction of siRNA into cells. The siRNA could be used to produce a change in a cell that can be therapeutic. The delivery of siRNA for therapeutic and research purposes is commonly called gene therapy.

A transfection reagent is a compound or compounds that bind(s) to or complex(es) with nucleic acid. The transfection reagent also mediates the binding and internalization of nucleic acid into cells. Examples of transfection reagents known in the art include cationic lipids and liposomes, polyamines, calcium phosphate precipitates, histone proteins, polyethylenimine, and polylysine complexes. Typically, the transfection reagent has a net positive charge that binds to the negative charge of the nucleic acid. The transfection reagent mediates binding to cells via its positive charge or via cell targeting signals that bind to receptors on or in the cell.

Functional groups include cell targeting signals (including nuclear localization signals), compounds that enhance release of contents from endosomes or other intracellular vesicles (releasing signals), and other compounds that alter the behavior or interactions of the compound or complex to which they are attached.

Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

After interaction of a compound or complex with the cell, other targeting groups can be used to increase the delivery of the biologically active compound to certain parts of the cell.

Nuclear localizing signals enhance the targeting of the pharmaceutical into proximity of the nucleus and/or its entry into the nucleus during interphase of the cell cycle. Such nuclear transport signals can be a protein or a peptide such as the SV40 large T antigen NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus. For example, karyopherin beta itself could target the DNA to the nuclear pore complex. Several peptides have been derived from the SV40 T antigen. Other NLS peptides have been derived from the hnRNP A1 protein, nucleoplasmin, c-myc, etc.

Many biologically active compounds, in particular large and/or charged compounds, are incapable of crossing biological membranes. In order for these compounds to enter cells, the cells must either take them up by endocytosis, i.e., into endosomes, or there must be a disruption of the cellular membrane to allow the compound to cross. In the case of endosomal entry, the endosomal membrane must be disrupted to allow for movement out of the endosome and into the cytoplasm. Either entry pathway into the cell requires a disruption of the cellular membrane. Compounds that disrupt or alter membranes or promote membrane fusion are called membrane active compounds. This change in structure can be shown by the compound inducing one or more of the following effects upon a membrane: an alteration that allows small molecule permeability, pore formation in the membrane, a fusion and/or fission of membranes, an alteration that allows large molecule permeability, or a dissolving of the membrane. This alteration can be functionally defined by the compound's activity in at least one the following assays: red blood cell lysis (hemolysis), liposome leakage, liposome fusion, cell fusion, cell lysis and endosomal release. An example of a membrane active agent in our examples is the peptide melittin, whose membrane activity is demonstrated by its ability to release heme from red blood cells (hemolysis). In addition, dimethylmaleamic-modified melittin reverts to melittin in the acidic environment of the endosome. More specifically membrane active compounds allow for the transport of molecules with molecular weight greater than 50 atomic mass units to cross a membrane. This transport may be accomplished by either the total loss of membrane structure, the formation of holes (or pores) in the membrane structure, or the assisted transport of compound through the membrane. In addition, transport between liposomes, or cell membranes, may be accomplished by the fusion of the two membranes and thereby the mixing of the contents of the two membranes. Membrane active compounds can be polymers, polyampholytes, peptides (such as cecropin, magainin, melittin, defensins, dermaseptin, hemagglutinin subunit HA-2 from influenza virus, EI from Semliki forest virus, HIV TAT peptide etc. as well as synthetic peptides) or small molecules (such as chloroquine, bafilomycin or Brefeldin A1). These membrane active compounds enhance release of endocytosed material from intracellular compartments such as endosomes (early and late), lysosomes, phagosomes, vesicle, endoplasmic reticulum, golgi apparatus, trans golgi network (TGN), and sarcoplasmic reticulum. Release includes movement out of an intracellular compartment into the cytoplasm or into an organelle such as the nucleus.

An interaction modifier changes the way that a molecule interacts with itself or other molecules, relative to molecule containing no interaction modifier. The result of this modification is that self-interactions or interactions with other molecules are either increased or decreased. Polyethylene glycol is an interaction modifier that decreases interactions between molecules and themselves and with other molecules. Dimethyl maleic anhydride modification or carboxy dimethylmaleic anhydride modification are other examples of interaction modifiers. Such groups can be useful in limiting interactions such as between serum factors and the molecule or complex to be delivered. They may also reversibly inhibit or mask an activity or function of a compound.

A labile bond is a covalent bond that is capable of being selectively broken. That is, the labile bond may be broken in the presence of other covalent bonds without the breakage of other covalent bonds. For example, a disulfide bond is capable of being broken in the presence of thiols without cleavage of any other bonds, such as carbon-carbon, carbon-oxygen, carbon-sulfur, carbon-nitrogen bonds, which may also be present in the molecule. Labile also means cleavable.

A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals. The spacer group may contain chemical moieties chosen from a group that includes alkanes, alkenes, esters, ethers, glycerol, amide, saccharides, polysaccharides, and heteroatoms such as oxygen, sulfur, or nitrogen. The spacer may be electronically neutral, may bear a positive or negative charge, or may bear both positive and negative charges with an overall charge of neutral, positive or negative.

pH-labile refers to the selective breakage of a covalent bond under acidic conditions (pH<7). That is, the pH-labile bond may be broken under acidic conditions without the breakage of other covalent bonds. The term pH-labile includes both linkages and bonds that are pH-labile, very pH-labile, and extremely pH-labile.

A subset of pH-labile bonds is very pH-labile. For the purposes of the present invention, a bond is considered very pH-labile if the half-life for cleavage at pH 5 is less than 45 minutes.

A subset of pH-labile bonds is extremely pH-labile. For the purposes of the present invention, a bond is considered extremely pH-labile if the half-life for cleavage at pH 5 is less than 15 minutes.

Linkages. A Linkage is an attachment that provides a covalent bond or spacer between two other groups (chemical moieties). The linkage may be electronically neutral, or may bear a positive or negative charge. The chemical moieties can be hydrophilic or hydrophobic. Preferred spacer groups include, but are not limited to C1-C12 alkyl, C1-C12 alkenyl, C1-C12 alkynyl, C6-C18 aralkyl, C6-C18 aralkenyl, C6-C18 aralkynyl, ester, ether, ketone, alcohol, polyol, amide, amine, polyglycol, polyether, polyamine, thiol, thio ether, thioester, phosphorous containing, and heterocyclic. The linkage may or may not contain one or more labile bonds.

pH-titratable groups (i.e. groups titratable at physiological pH) are chemical functional groups that lose or gain a proton in aqueous solution in the pH range 4-8. Groups titratable at physiological pH act as buffers within the pH range of 4-8. Groups titratable at physiological pH can be determined experimentally by conducting an acid-base titration and experimentally determining if the group buffers within the pH-range of 4-8. Examples of chemical functional groups that can exhibit buffering within this pH range include but are not limited to carboxylic acids, imidazole, N-substituted imidazole, pyridine, phenols, and polyamines. Groups titratable at physiological pH can include polymers, non-polymers, peptides, modified peptides, proteins, and modified proteins.

An RNA function inhibitor comprises any nucleic acid or nucleic acid analog containing a sequence whose presence or expression in a cell causes the degradation of or inhibits the function or translation of a specific cellular RNA, usually a mRNA, in a sequence-specific manner. Inhibition of RNA can thus effectively inhibit expression of a gene from which the RNA is transcribed. RNA function inhibitors are selected from the group comprising: siRNA, interfering RNA or RNAi, dsRNA, RNA Polymerase III transcribed DNAs, ribozymes, and antisense nucleic acid, which may be RNA, DNA, or artificial nucleic acid. SiRNA comprises a double stranded structure typically containing 15-50 base pairs and preferably 21-25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The RNA function inhibitor may be polymerized in vitro, recombinant RNA, contain chimeric sequences, or derivatives of these groups. The RNA function inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

The term nucleic acid, or polynucleotide, is a term of art that refers to a polymer containing at least two nucleotides. Nucleotides are the monomeric units of nucleic acid polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA).

An activated carboxylate is a carboxylic acid derivative that reacts with nucleophiles to form a new covalent bond. Nucleophiles include nitrogen, oxygen and sulfur-containing compounds to produce ureas, amides, carbonates, carbamates, esters, and thioesters. The carboxylic acid may be activated by various agents including carbodiimides, carbonates, phosphoniums, and uroniums to produce activated carboxylates acyl ureas, acylphosphonates, acid anhydrides, and carbonates. Activation of carboxylic acid may be used in conjunction with hydroxy and amine-containing compounds to produce activated carboxylates N-hydroxysuccinimide esters, hydroxybenzotriazole esters, N-hydroxy-5-norbornene-endo-2,3-dicarboximide esters, p-nitrophenyl esters, pentafluorophenyl esters, 4-dimethylaminopyridinium amides, and acyl imidazoles.

Alkyl means any sp³-hybridized carbon-containing group; alkenyl means containing two or more Sp² hybridized carbon atoms; aklkynyl means containing two or more sp hybridized carbon atoms; aryl means containing one or more aromatic ring(s) (including heterocyclic aromatic rings), aralkyl means containing one or more aromatic ring(s) in addition containing sp³ hybridized carbon atoms; aralkenyl means containing one or more aromatic ring(s) in addition to containing two or more sp² hybridized carbon atoms; aralkynyl means containing one or more aromatic ring(s) in addition to containing two or more sp hybridized carbon atoms; steroid includes natural and unnatural steroids and steroid derivatives.

Amphipathic, or amphiphilic, compounds have both hydrophilic (water-soluble) and hydrophobic (water-insoluble) parts. Hydrophilic groups indicate in qualitative terms that the chemical moiety is water-preferring. Typically, such chemical groups are water soluble, and are hydrogen bond donors or acceptors with water. Examples of hydrophilic groups include compounds with the following chemical moieties; carbohydrates, polyoxyethylene, peptides, oligonucleotides and groups containing amines, amides, alkoxy amides, carboxylic acids, sulfurs, or hydroxyls. Hydrophobic groups indicate in qualitative terms that the chemical moiety is water-avoiding. Typically, such chemical groups are not water soluble, and tend not to form hydrogen bonds. Hydrocarbons are hydrophobic groups.

Bifunctional molecules, commonly referred to as crosslinkers, are used to connect two molecules together, i.e. form a linkage between two molecules. Bifunctional molecules can contain homo or heterobifunctionality.

A chelator is a polydentate ligand, a molecule that can occupy more than one site in the coordination sphere of an ion, particularly a metal ion, primary amine, or single proton. Examples of chelators include crown ethers, cryptates, and non-cyclic polydentate molecules. A crown ether is a cyclic polyether containing (—X—(CR1-2)n)m units, where n=1-3 and m=3-8. The X and CR1-2 moieties can be substituted, or at a different oxidation states. X can be oxygen, nitrogen, or sulfur, carbon, phosphorous or any combination thereof. R can be H, C, O, S, N, P. A subset of crown ethers described as a cryptate contain a second (—X—(CR1-2)n)z strand where z=3-8. The beginning X atom of the strand is an X atom in the (—X—(CR1-2)n)m unit, and the terminal CH2 of the new strand is bonded to a second X atom in the (—X—(CR1-2)n)m unit. Non-cyclic polydentate molecules containing (—X—(CR1-2)n)m unit(s), where n=1-4 and m=1-8. The X and CR1-2 moieties can be substituted, or at a different oxidation states. X can be oxygen, nitrogen, or sulfur, carbon, phosphorous or any combination thereof.

A polychelator is a polymer associated with a plurality of chelators by an ionic or covalent bond and can include a spacer. The polymer can be cationic, anionic, zwitterionic, neutral, or contain any combination of cationic, anionic, zwitterionic, or neutral groups with a net charge being cationic, anionic or neutral, and may contain steric stabilizers, peptides, proteins, signals, or amphipathic compound for the formation of micellar, reverse micellar, or unilamellar structures. Preferably the amphipathic compound can have a hydrophilic segment that is cationic, anionic, or zwitterionic, and can contain polymerizable groups, and a hydrophobic segment that can contain a polymerizable group.

Two molecules are combined, to form a complex through a process called complexation or complex formation, if the are in contact with one another through noncovalent interactions such as coordination bonds, electrostatic interactions, hydrogen bonding interactions, and hydrophobic interactions.

Derivative, or substructure, means the chemical structure of the compound and any compounds derived from that chemical structure from the replacement of one or more hydrogen atoms by any other atom or change in oxidation state. For example if the substructure is succinic anhydride, then methylsuccinic anhydride, 2,2-dimethylsuccinic anhydride, 3-oxabicyclo[3.1.0]hexane-2,4-dione, maleic anhydride, citriconic anhydride, and 2,3-dimethylmaleic anhydride have the same substructure, or are derivatives. In the same way, derivatives of maleic anhydride include: methyl maleic anhydride, citraconic anhydride, dimethyl maleic anhydride, and 2-propionic-3-methylmaleic anhydride.

A molecule is modified, to form a modification through a process called modification, by a second molecule if the two become bonded through a covalent bond. That is, the two molecules form a covalent bond between an atom from one molecule and an atom from the second molecule resulting in the formation of a new single molecule. A chemical covalent bond is an interaction, bond, between two atoms in which there is a sharing of electron density.

Hydrophobic stabilization means the stability gained in a complex in water due to the noncovalent interactions between hydrophobic groups in the system.

A lipid is any of a diverse group of organic compounds that are insoluble in water, but soluble in organic solvents such as chloroform and benzene. Lipids contain both hydrophobic and hydrophilic sections. Lipids is meant to include complex lipids, simple lipids, and synthetic lipids.

Simple lipids include steroids and terpenes.

Complex lipids are the esters of fatty acids and include glycerides (fats and oils), glycolipids, phospholipids, and waxes.

Phospolipids are lipids having both a phosphate group and one or more fatty acids (as esters of the fatty acid). The phosphate group may be bound to one or more additional organic groups.

Glycolipids are sugar containing lipids. The sugars are typically galactose, glucose or inositol.

A steroid derivative means a sterol, a sterol in which the hydroxyl moiety has been modified (for example, acylated), or a steroid hormone, or an analog thereof. The modification can include spacer groups, linkers, or reactive groups.

Synthetic lipids includes amides prepared from fatty acids wherein the carboxylic acid has been converted to the amide, synthetic variants of complex lipids in which one or more oxygen atoms has been substituted by another heteroatom (such as Nitrogen or Sulfur), and derivatives of simple lipids in which additional hydrophilic groups have been chemically attached. Synthetic lipids may contain one or more labile group.

Peptide and polypeptide refer to a series of amino acid residues, more than two, connected to one another by amide bonds between the beta or alpha-amino group and carboxyl group of contiguous amino acid residues. The amino acids may be naturally occurring or synthetic. Polypeptide includes proteins and peptides, modified proteins and peptides, and non-natural proteins and peptides. Bioactive compounds may be used interchangeably with biologically active compound for purposes of this application.

A compound is reactive if it is capable of forming either an ionic or a covalent bond with another compound. The portions of reactive compounds that are capable of forming covalent bonds are referred to as reactive functional groups.

A salt is any compound containing ionic bonds, (i.e., bonds in which one or more electrons are transferred completely from one atom to another). Salts are ionic compounds that dissociate into cations and anions when dissolved in solution and thus increase the ionic strength of a solution. Pharmaceutically acceptable salt means both acid and base addition salts.

Pharmaceutically acceptable acid addition salts are those salts which retain the biological effectiveness and properties of the free bases, and are not biologically or otherwise undesirable, and which are formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethansulfonic acid, p-toluenesulfonic acid, salicylic acid, trifluoroacetic acid, and the like.

Pharmaceutically acceptable base addition salts are those salts which retain the biological effectiveness and properties of the free acids, and are not biologically or otherwise undesirable. The salts are prepared from the addition of an inorganic base or an organic base to the free acid. Salts derived from inorganic bases include, but are not limited to, sodium, potassium, calcium, lithium, ammonium, magnesium, zinc, and aluminum salts and the like. Salts derived from organic bases include, but are not limited to salts of primary secondary, and tertiary amines, such as methylamine, triethylamine, and the like.

Steric hindrance, or sterics, is the prevention or retardation of a chemical reaction because of neighboring groups on the same molecule.

A steric stabilizer is a long chain hydrophilic group that prevents aggregation of final polymer by sterically hindering particle to particle electrostatic interactions. Examples include: alkyl groups, PEG chains, polysaccharides, alkyl amines. Electrostatic interactions are the non-covalent association of two or more substances due to attractive forces between positive and negative charges.

A substituted group or a substitution refers to chemical group that is placed onto a parent system instead of a hydrogen atom. For the compound methylbenzene (toluene), the methyl group is a substituted group, or substitution on the parent system benzene. The methyl groups on 2,3-dimethylmaleic anhydride are substituted groups, or substitutions on the parent compound (or system) maleic anhydride.

The foregoing is considered as illustrative only of the principles of the invention. Further, since numerous modifications and changes will readily occur to those skilled in the art, it is not desired to limit the invention to the exact construction and operation shown and described. Therefore, all suitable modifications and equivalents fall within the scope of the invention.

### EXAMPLES

### Example 1

### Synthesis of Polyampholytes

A. Branched PEI (brPEI)-polyGlutamic acid (pGlu) and brPEI-polyAspartic acid (pAsp) pGlu (2.28 mg in 172 μl of water, pH 5.5) or pAsp (2 mg in 172 μL of water) were activated in the presence of 100 μg of EDC and N-hydroxysulfosuccinimide (Sulfo-NHS) each for 10 min at RT. BrPEI (4 mg) and 2.5 M Na Cl (0.5 ml) solutions were added to the activated polyanion. The reaction mixture was allowed to incubate for 5 h at RT. Resulting brPEI-based polyampholytes were dialyzed against water and freeze-dried.

B. Linear PEI (lPEI)-poly(Methacrylic acid) pMAA and lPEI-pGlu

The following polyions were used for the reaction: lPEI (Mw=25 kDa, Polysciences), pMAA (MW=9.5 kDa, Aldrich), pGlu (MW=49 kDa, Sigma). For analytical purposes polyanions covalently labeled with rhodamine-ethylenediamine (Molecular Probes) were used for these reactions (degree of carboxy group modification <2%). pMAA (1 mg in 100 μL water) was activated in the presence of water-soluble carbodiimide (EDC, 100 μg) and Sulfo-NHS (100 μg) for 10 min at pH 5.5. Activated pMAA was added to the solution of lPEI (2 mg in 200 μL of 25 mM HEPES, pH 8.0) and incubated for 1 b at RT. pGlu was used at the same molar ratio.

After reaction completion, an equal volume of 3 M NaCl solution was added to a part of the reaction mixture. This part (0.5 ml) was passed through a Sepharose 4B-CL column (1×25 cm) equilibrated in 1.5 M NaCl and 1 ml fractions were collected. Rhodamine fluorescence was measured in each fraction. lPEI was measured using fluorescamine reaction. The amount of polyampholyte in the lPEI-pGlu reaction mixture was about 50%.

C. Melittin-pGlu (Partially Esterified with Di-(2-methyl-4-hydroxymethyl-1,3-dioxolane)-1,4-benzene). To a solution of the aldehyde-poly-glutamic acid compound (1.0 mg, 7.7 μmol) in water (200 μL) was added melittin (4.0 mg, 1.4 μmol) and the reaction mixture was stirred at RT for 12 h. The reaction mixture was then divided into two equal portions. One sample (100 μL) was dialyzed against 1% ethanol in water (2×1 L, 12,000-14,000 MWCO) and tested utilizing a theoretical yield of 1.7 mg. To the second portion (100 μL) was added sodium cyanoborohydride (1.0 mg, 16 μmol, Aldrich Chemical Company). The solution was stirred at RT for 1 h and then dialyzed against water (2×1 L, 12,000-14,000 MWCO).

D. CDM-DW297-DW301 pH-labile polyampholyte formed in the presence of DNA. Polycation DW297, was modified into a pH-labile polyanion by reaction with a 4 weight excess of CDM aldehyde in the presence of 25-fold weight equivalents HEPES base. DNA (10 μg/mL) in 5 mM HEPES pH 7.5 was condensed by the addition of polycation DW301 (10 μg/mL). To the polycation-DNA particle was added the aldehyde-containing, pH-labile polyanion derived from DW297 (30 μg/mL). Particles formulated in this manner are 100-130 nm in size and are stable in 150 mM NaCl. The stability of particle size indicates that a covalent bond between the polycation and the polyanion of the complex has formed via an imine bond. In other words, the aldehyde of the polyanion has formed a bond with polycation, which results in the formation of a polyampholyte.

E. DM-KL₃-PLL, 2-propionic-3-methylmaleamic (CDM)-KL₃-PLL, and succinylated KL₃-PLL. See example 2D.

F. Poly-L-Glutamic acid (octamer)-Glutaric Dialdehyde Copolymer (MC151): H₂N-EEEEEEEE-NHCH₂CH₂NH₂ (5.5 mg, 0.0057 mmol, Genosis) was taken up in 0.4 mL H₂O. Glutaric dialdehyde (0.52 μL, 0.0057 mmol, Aldrich Chemical Company) was added and the mixture was stirred at RT. After 10 min the solution was heated to 70° C. After 15 h, the solution was cooled to RT and dialyzed against H₂O (2×2 L, 3500 MWCO). Lyophilization afforded 4.3 mg (73%) poly-glutamic acid (octamer)-glutaric dialdehyde copolymer.

G. poly N-terminal acryloyl 6-aminohexanoyl-KLLKLLLKLWLKLLKLLLKLL-CO2 (pAcKL₃): A solution of AcKL3 (20 mg, 7.7 mmol) in 0.5 mL of 6M guanidinium hydrochloride, 2 mM EDTA, and 0.5 M Tris pH 8.3 was degassed by placing under a 2 torr vacuum for 5 minutes. Polymerization of the acrylamide was initiated by the addition of ammonium persulfate (35 μg, 0.02 eq.) and N,N,N,N-tetramethylethylenediamine (1 μL). The polymerization was allowed to proceed overnight. The solution was then placed into dialysis tubing (12,000 molecular weight cutoff) and dialyzed against 3×2 L over 48 b. The amount of polymerized peptide (6 mg, 30% yield) was determined by measuring the absorbance of the tryptophan residue at 280 nm, using an extinction coefficient of 5690 cm⁻¹ M⁻¹ [Gill S C and von Hippel P H 1989].

H. pH-labile polyampholytes using CDM-thioester and cysteine-modified polyeations: A pH-labile polyanion is generated by the reaction of a polyamine with 2 equivalents (relative to amines) of CDM thioester. A cysteine-modified polycation is deprotected by reduction of disulfide with dithiothreitol. The thioester-containing, pH-labile polyanion is added to the cysteine-modified polycation. The thioester groups and cysteine groups react to produce a pH-labile polyampholyte. Polyeations that can modified with cysteine and used as pH-labile polyanion may be selected from the group comprising: PLL, polyallylamine, polyvinylamine, polyethyleneimine, and histone H1.

I. A method for synthesizing such a polyampholyte is to react amine-containing compounds with poly (methylvinylether maleic anhydride) pMVMA. The anhydride of pMVMA reacts with amines to form an amide and an acid. Two different amine and imidazole containing compounds were used: histidine, which also attaches a carboxylic acid group, and histamine which just attaches an imidazole group. The histidine containing polymer (MC#486) and the histamine containing polymer (MC#510) are alternating copolyampholytes.

MC510: To a solution of poly(methyl vinyl ether-alt-maleic anhydride) (purchased from Aldrich Chemical) 50 mg in 10 mL of anhydrous tetrahydrofuran was added 100 mg of histamine. The solution was stirred for 1 h followed by the addition of 10 mL water. The solution was stirred for another hour and then placed into a 12,000 MW cutoff dialysis tubing and dialyzed against 7×4 L water over a one week period. The solution was then removed from the dialysis tubing and then concentrated to 1 mL volume by lyophilization.

MC486: To a solution of histidine (150 mg) and potassium carbonate (150 mg) in 10 mL water was added 50 mg of poly(methyl vinyl ether-alt-maleic anhydride) (purchased from Aldrich Chemical). The solution was stirred for 1 h and then placed into a 12,000 MW cutoff dialysis tubing and dialyzed against 7×4 L water over a one week period. The solution was then removed from the dialysis tubing and then concentrated to 1 mL volume by lyophilization.

To determine the effect of pH on these MC510 and MC486, we measured the amount of polymer needed to condense fluorescein-labeled polylysine at pH 7.5 and pH 6.0. As fluorescein-labeled polylysine is condensed by addition of a negatively charged polyelectrolyte, the fluorescein fluorophores are brought closer together, causing fluorescence to be quenched. This quenching enables one to measure the extent of condensation and thus the charge density of the polyelectrolyte. The histamine containing polymer, MC#510, required significantly more material to condense the polylysine at pH 6.0 than at pH 7.5. Approximately five-fold more polymer was required. The histidine-containing polymer, MC#486, also need more material at pH 6.0, approximately two-fold more. These data suggest that we have made polyanions which are pH-sensitive in a pH range that is important for endosomal release.

J. Polyallylamine-graft imidazoleacetic acid polycation (DW163): Polyallylamine (15,000 MW) is dissolved to 50 mg/mL in 100 mM MES (pH 6.5) buffer in a 15-ml polypropylene tube. To this solution is added 1.1 molar equivalent (relative to amine content of polyallylamine) of 4-imidazoleacetic acid. N-(3-Dimethylaminopropyl)-N′-ethylcarbodiimide (EDC) (1.1 equivalent) and N-hydroxysuccinimide (1.1 equivalent) are dissolved in 2 ml of MES buffer and are added immediately to the polyallylamine solution. The reaction tube was sealed and allowed to react at RT for 24 h. The reaction mixture is then removed from tube and placed into dialysis tubing (3,500 MW cutoff), and dialyzed against 7×4 L water over a one week period. The polymer is then removed from the tubing and concentrated by lyophilization to 10 mg/mL.

K. MC750: To a solution of poly(methyl vinyl ether-alt-maleic anhydride) (purchased from Aldrich Chemical) 50 mg in 10 mL of anhydrous tetrahydrofuran was added 100 mg of 1-(3-aminopropyl)imidazole. The solution was stirred for 1 h followed by the addition of 10 mL water. The solution was stirred for another hour and then placed into a 12,000 MW cutoff dialysis tubing and dialyzed against 7×4 L water over a one week period. The solution was then removed from the dialysis tubing and then concentrated to 1 mL volume by lyophilizati on.

L. Acetal-containing polyampholyte DW 179A and DW 179B: To a solution of poly(methyl vinyl ether-alt-maleic anhydride) (purchased from Aldrich Chemical) 20 mg in 5 mL of anhydrous tetrahydrofuran was added 1.4 or 3.5 μL of aminoacetaldhyde dimethyl acetal (0.01 or 0.025 mol eq.) and this solution was stirred for 3 h followed by the addition of 80 mg of histamine. The solution was then stirred for 24 h followed by the addition of 10 mL water. The solution was stirred for another hour and then placed into a 12,000 MW cutoff dialysis tubing and dialyzed against 7×4 L water over a one week period. The solution was then removed from the dialysis tubing and then concentrated to 1 mL volume by lyophilization. The polyampholyte containing 0.01 eq acetal was given the number DW#179A and the polyampholyte containing 0.025 eq acetal was given the number DW#179B. The acetal groups of DW#179 were removed to produce aldehyde groups by placing 1 mg of DW179 into 1 mL centrifuge tube, and adjusting the pH to 3.0 with 1M HCl and left at RT 12 h. After incubation at acidic pH, the DW# 179 may be added to polyamine-condensed DNA to form a Schiff between the amine and the aldehyde thus forming a polyampholyte.

M. Poly(Acrylic acid-co-maleic acid) graft Histamine Polymer (MC758): A solution of Poly(Acrylic acid-co-maleic acid)(0.050 g, 0.026 mmol), histamine (0.029 g, 0.026 mmol) were dissolved in 5 mL of 100 mM 2-[N-morpholino]ethanesulfonic acid (MES) at pH 6.5. This solution was then added to 1,[3-(dimethylamino)propyl]-3-ethylcarboimide(EDC)(0.057 g, 0.029 mmol), followed by the addition of N-hydroxysuccinimide(NHS)(0.033 g, 0.029 mmol) in 0.5 mL of pH 6.5 100 mM MES. This solution was sealed tightly and stirred for 24 h at RT. This solution was then transferred to 12,000 to 14,000 molecular weight tubing and dialyzed against distilled water for 4 days, and freeze dried.

N. Poly(Acrylic acid-co-maleic acid) graft 1-(3-amino-propyl)imidazole Polymer (MC757): Poly(Acrylic acid-co-maleic acid) (0.050 g, 0.026 mmol), and 1-(3-amino-propyl)imidazole (0.0155 g, 0.013 mmol) were dissolved in 5 mL of 100 MES at pH 6.5. This solution was then added to 1,[3-(dimethylamino)propyl]-3-ethylcarboimide (EDC, 0.0312 g, 0.016 mmol), followed by the addition of N-hydroxysuccinimide (NHS, 0.012 g, 0.016 mmol) in 0.5 mL of pH 6.5 100 mM MES. This solution was sealed tightly and stirred for 24 h at RT. This solution was then transferred to 12,000 to 14,000 molecular weight tubing and dialyzed against distilled water for 4 days, and freeze dried.

### Example 2

### Synthesis of Compounds Utilized in the Formation of Polyampholytes

A. 2-propionic-3-methylmaleic anhydride (carboxydimethylmaleic anhydride or CDM): To a suspension of sodium hydride (0.58 g, 25 mmol) in 50 mL anhydrous tetrahydrofuran was added triethyl-2-phosphonopropionate (7.1 g, 30 mmol). After bubbling of hydrogen gas stopped, dimethyl-2-oxoglutarate (3.5 g, 20 mmol) in 10 mL anhydrous tetrahydrofuran was added and stirred for 30 minutes. Water, 10 mL, was then added and the tetrahydrofuran was removed by rotary evaporation. The resulting solid and water mixture was extracted with 3×50 mL ethyl ether. The ether extractions were combined, dried with magnesium sulfate, and concentrated to a light yellow oil. The oil was purified by silica gel chromatography elution with 2:1 ether:hexane to yield 4 gm (82% yield) of pure triester. The 2-propionic-3-methylmaleic anhydride then formed by dissolving of this triester into 50 mL of a 50/50 mixture of water and ethanol containing 4.5 g (5 equivalents) of potassium hydroxide. This solution was heated to reflux for 1 h. The ethanol was then removed by rotary evaporation and the solution was acidified to pH 2 with hydrochloric acid. This aqueous solution was then extracted with 200 mL ethyl acetate, which was isolated, dried with magnesium sulfate, and concentrated to a white solid. This solid was then recrystallized from dichloromethane and hexane to yield 2 g (80% yield) of 2-propionic-3-methylmaleic anhydride.

B. 2,3-dioleoyldiaminopropionic ethylenediamine amide: 2,3-diaminopropionic acid (1.4 gm, 10 mmol) and dimethylaminopyridine (1.4 gm 11 mmol) were dissolved in 50 mL of water. To this mixture was added over 5 minutes with rapid stirring olcoyl chloride (7.7 mL, 22 mmol) of in 20 mL of tetrahydrofuran. After all of the acid chloride had been added, the solution was allowed to stir for 30 minutes. The pH of the solution was 4 at the end of the reaction. The tetrahydrofuran was removed by rotary evaporation. The mixture was then partitioned between water and ethyl acetate. The ethyl acetate was isolated, dried with magnesium sulfate, and concentrated by rotary evaporation to yield a yellow oil. The 2,3-dioleoyldiaminopropionic acid was isolated by silica gel chromatography, elution with ethyl ether to elute oleic acid, followed by 10% methanol 90% methylene chloride to elute diamide product, 1.2 g (19% yield). The diamide (1.1 gm, 1.7 mmol) was then dissolved in 25 mL of methylene chloride. To this solution was added N-hydroxysuccinimide (0.3 g. 1.5 eq) and dicyclohexylcarbodiimide (0.54 g, 1.5 eq). This mixture was allowed to stir overnight. The solution was then filtered through a cellulose plug. To this solution was added ethylene diamine (1 gm, 10 eq) and the reaction was allowed to proceed for 2 h. The solution was then concentrated by rotary evaporation. The resulting solid was purified by silica gel chromatography elution with 10% ammonia saturated methanol and 90% methylene chloride to yield the triamide product 2,3-dioleoyldiaminopropionic ethylenediamine amide (0.1 gm, 9% yield). The triamide product was given the number MC213.

C. Dioleylamideaspartic acid: N-(tert-butoxycarbonyl)-L-aspartic acid (0.5 gm, 2.1 mmol) was dissolved in 50 mL of acetonitrile. To this solution was added N-hydroxysuccinimide (0.54 gm, 2.2 eq) and was added dicyclohexylcarbodiimide (0.54 g, 1.5 eq). This mixture was allowed to stir overnight. The solution was then filtered through a cellulose plug. This solution was then added over 6 h to a solution containing oleylamine (1.1 g, 2 eq) in 20 mL methylene chloride. After the addition was complete the solvents were removed by rotary evaporation. The resulting solid was partitioned between 100 mL ethyl acetate and 100 mL water. The ethyl acetate fraction was then isolated, dried by sodium sulfate, and concentrated to yield a white solid. The solid was dissolved in 10 mL of triflouroacetic acid, 0.25 mL water, and 0.25 mL triisopropylsilane. After two h, the triflouroacetic acid was removed by rotary evaporation. The product was then isolated by silica gel chromatography using ethyl ether followed by 2% methanol 98% methylene chloride to yield 0.1 gm (10% yield) of pure dioleylamideaspartic acid, which was given the number MC303.

D. Dimethylmaleamic-peptides: Solid melittin or pardaxin or other peptide (100 μg) was dissolved in 100 μL of anhydrous dimethylformamide containing 1 mg of 2,3-dimethylmaleic anhydride and 6 μL of diisopropylethylamine. Similar procedures were used for derivatives of dimethylmaleic anhydride such as 2-propionic-3-methylmaleic anhydride (CDM) and CDM-thioester.

E. Polyethyleneglycol methyl ether 2-propionic-3-methylmaleate (CDM-PEG): To a solution of 2-propionic-3-methylmaleic anhydride (30 mg, 0.16 mmol) in 5 mL methylene chloride was added oxalyl chloride (200 mg, 10 eq) and dimethylformamide (1 μL). The reaction was allowed to proceed overnight at which time the excess oxalyl chloride and methylene chloride were removed by rotary evaporation to yield the acid chloride, a clear oil. The acid chloride was dissolved in 1 mL of methylene chloride. To this solution was added polyethyleneglycol monomethyl ether, molecular weight average of 5,000 (815 mg, 1 eq) and pyridine (20 μL, 1.5 eq) in 10 mL of methylene chloride. The solution was then stirred overnight. The solvent was then removed and the resulting solid was dissolved into 8.15 mL of water.

F. Polyvinyl(2-phenyl-4-hydroxymethyl-1,3-dioxolane) from the reaction of Polyvinylphenyl Ketone and Glycerol: Polyvinyl phenyl ketone (500 mg, 3.78 mmol, Aldrich Chemical Company) was taken up in 20 mL dichloromethane. Glycerol (304 μL, 4.16 mmol, Acros Chemical Company) was added followed by p-toluenesulfonic acid monohydrate (108 mg, 0.57 mmol, Aldrich Chemical Company). Dioxane (10 mL) was added and the solution was stirred at RT overnight. After 16 h, TLC indicated the presence of ketone. The solution was concentrated under reduced pressure, and the residue dissolved in dimethylformamide (7 mL). The solution was heated to 60° C. for 16 h. After 16 h, TLC indicated the ketone had been consumed. Dialysis against H₂O (1×3 L, 3500 MWCO), followed by lyophilization resulted in 606 mg (78%) of the ketal. Ketone was not observed in the sample by TLC analysis, however, upon treatment with acid, the ketone was again detected.

G. Peptide synthesis: Peptide syntheses were performed using standard solid phase peptide techniques using FMOC chemistry.

H. Coupling KL₃ to poly(allylamine): To a solution of poly(allylamine) (2 mg) in water (0.2 mL) was added KL₃ (0.2 mg, 2.5 eq) and 1-(3-dimethylaminopropyl)-3-ethylcarbodiimide hydrochloride (1 mg, 150 eq). The reaction was allowed to react for 16 h and then the mixture was placed into dialysis tubing and dialyzed against 3×1 L for 48 h. The solution was then concentrated by lyophilization to 0.2 mL.

I. Aldehyde adduct of 2-propionic-3-methylmaleic anhydride (CDM-aldehyde): To a solution of 2-propionic-3-methylmaleic anhydride (CDM) 50 mg in 5 mL methylene chloride was added 1 mL oxalyl chloride. The solution was stirred overnight at RT. The excess oxalyl chloride and methylene chloride was removed by rotary evaporation to yield a clear oil. The oil was then dissolved in methylene chloride (5 mL) and 85 mg of 2,2-dimethoxyethylamine was added. The solution was added to proceed for 1 h. The solvent was removed by rotary evaporation to yield a yellow oil which was placed under high vacuum (1 torr) for 24 h. The resulting oil was dissolved in 5 mL water and chromatographed by reverse-phase HPLC eluting with acetonitrile containing 0.1% trifluoroacetic acid to produce the dimethyl acetal (20 mg). To remove the acetal, it was dissolved in 1 mL acetonitrile and 0.1 mL concentrated hydrochloric acid. The aldehyde was isolated by reverse-phase HPLC eluting with acetonitrile containing 0.1% trifluoroacetic acid to produce 10 mg of aldehyde adduct of 2-propionic-3-methylmaleic anhydride (CDM-aldehyde).

J. Mercaptoacetic acid thioester of 2-propionic-3-methylmaleic anhydride (CDM thioester): To a solution of 2-propionic-3-methylmaleic anhydride (CDM) 50 mg in 5 mL methylene chloride was added 1 mL oxalyl chloride. The solution was stirred overnight at RT. The excess oxalyl chloride and methylene chloride was removed by rotary evaporation to yield a clear oil. The oil was then dissolved in methylene chloride (5 mL) and 25 mg of mercaptoacetic acid was added, followed by the addition of 70 mg of diisopropylethylamine. After 1 h, the solvent was removed by rotary evaporation and excess mercaptoacetic acid and diisopropylethylamine were removed by placing the sample under high vacuum (1 torr) for 24 h. The resulting oil was dissolved in 5 mL water and chromatographed by reverse-phase HPLC eluting with acetonitrile containing 0.1% trifluoroacetic acid to produce the thioester.

### Example 3

### Synthesis of Acid Labile Monomers

A. Di-(2-methyl-4-hydroxymethyl-1,3-dioxolane)-1,4-benzene (MC216).

To a solution of diacetylbenzene (2.00 g, 12.3 mmol, Aldrich Chemical Company) in toluene (30.0 mL), was added glycerol (5.50 g, 59.7 mmol, Acros Chemical Company) followed by p-toluenesulfonic acid monohydrate (782 mg, 4.11 mmol, Aldrich Chemical Company). The reaction mixture was heated at reflux for 5 h with the removal of water by azeotropic distillation in a Dean-Stark trap. The reaction mixture was concentrated under reduced pressure, and the residue was taken up in Ethyl Acetate. The solution was washed 1× with 10% NaHCO₃, 3× with H₂O, 1× with brine, and dried (MgSO₄). Following removal of solvent (aspirator), the residue was purified by flash chromatography on silica gel (20×150 mm, CH₂Cl₂ eluent) to afford 593 mg (16% yield) of di-(2-methyl-4-hydroxymethyl-1,3-dioxolane)-1,4-benzene. Molecular ion calculated for C₁₆H₂₂O₆ 310, found m+1/z 311.2; 300 MHz NMR (CDCl₃, ppm) δ 7.55-7.35 (4H, m) 4.45-3.55 (10H, m) 1.65 (6H, brs).

B. Di-(2-methyl-4-hydroxymethyl(succinic semialdehyde ester)-1,3-dioxolane)-1,4-benzene (MC 211): To a solution of succinic semialdehyde (150 mg, 1.46 mmol, Aldrich Chemical Company) and di-(2-methyl-4-hydroxymethyl-1,3-dioxolane)-1,4-benzene (150 mg, 480 μmol) in CH₂Cl₂ (4 mL) was added dicyclohexylcarbodiimide (340 mg, 1.65 mmol, Aldrich Chemical Company) followed by a catalytic amount of 4-dimethylaminopyridine. The solution was stirred for 30 min and filtered. Following removal of solvent (aspirator), the residue was purified by flash chromatography on silica gel (20×150 mm, CH₂Cl₂ eluent) to afford 50 mg (22%) of di-(2-methyl-4-hydroxymethyl(succinic semialdehyde ester)-1,3-dioxolane)-1,4-benzene. Molecular ion calculated for C₂₄H₃₀O₁₀ 478.0 found m+1/z 479.4.

C. Di-(2-methyl-4-hydroxymethyl(glyoxilic acid ester)-1,3-dioxolane)-1,4-benzene (MC225): To a solution of glyoxylic acid monohydrate (371 mg, 403 μmol, Aldrich Chemical Company) and di-(2-methyl-4-hydroxymethyl-1,3-dioxolane)-1,4-benzene (500 mg, 161 μmol) in dimethylformamide (8 mL) was added dicyclohexylcarbodiimide (863 mg, 419 μmol, Aldrich Chemical Company). The solution was stirred for 30 min and filtered. Following removal of solvent (aspirator), the residue was purified by flash chromatography on silica gel (20×150 mm, ethylacetate/Hexanes (1:2.3 eluent) to afford 58 mg (10%) of di-(2-methyl-4-hydroxymethyl(glyoxylic acid ester)-1,3-dioxolane)-1,4-benzene.

D. Synthesis of Di-(2-methyl-4-aminomethyl-1,3-dioxolane)-1,4-benzene (MC372): To a solution of 1,4-diacetylbenzene (235 mg, 1.45 mmol, Aldrich Chemical Company) in toluene (15.0 mL) was added 3-amino-1,2-propanediol protected as the FMOC carbamide (1.0 g, 3.2 mmol), followed by a catalytic amount of p-toluenesulfonic acid monohydrate (Aldrich Chemical Company). The reaction mixture was heated at reflux for 16 h with the removal of water by azeotropic distillation in a Dean-Stark trap. The reaction mixture was cooled to RT, partitioned in toluene/H₂O, washed 1×10% NaHCO₃, 3×H₂O, 1× brine, and dried (MgSO₄). The extract was concentrated under reduced pressure and crystallized (methanol/H₂O). The protected amine ketal was identified in the supernatant, which was concentrated to afford 156 mg product. The free amine was generated by treating the ketal with piperidine in dichloromethane for 1 h.

E. Di-(2-methyl-4-hydroxymethyl(glycine ester)-1,3-dioxolane)-1,4-benzene (MC373): To a solution of FMOC-Glycine (690 mg, 2.3 mmol, NovaBiochem) in dichloromethane (4.0 mL) was added dicyclohexylcarbodiimide (540 mg, 2.6 mmol, Aldrich Chemical Company). After 5 min, di-(2-methyl-4-hydroxymethyl-1,3-dioxolane)-1,4-benzene (240 mg, 770 μmol) was added followed by a catalytic amount of 4-dimethylaminopyridine. After 20 min, the reaction mixture was filtered and concentrated (aspirator) to afford 670 mg oil. The residue was taken in tetrahydrofuran (4.0 mL) and piperidine (144 mg, 1.7 mmol) was added. The reaction was stirred at RT for 1 h and added to cold diethyl ether. The resulting solid was washed 3× diethyl ether to afford di-(2-methy-4-hydroxymethyl(glycine ester)-1,3-dioxolane)-1,4,benzene. Molecular ion calculated for C₂₀H₂₈N₂O₈ 424, found m+1/z 425.2.

### Example 4

### Synthesis of Polyanions

A. 2,3-dimethylmaleamic poly-L-lysine: Poly-L-lysine (10 mg 34,000 MW Sigma Chemical) was dissolved in 1 mL of aqueous potassium carbonate (100 mM). To this solution was added 2,3-dimethylmaleic anhydride (100 mg, 1 mmol) and the solution was allowed to react for 2 h. The solution was then dissolved in 5 mL of aqueous potassium carbonate (100 mM) and dialyzed against 3×2 L water that was at pH8 with addition of potassium carbonate. The solution was then concentrated by lyophilization to 10 mg/mL of 2,3-dimethylmaleamic poly-L-lysine.

B. Melittin-PAA, KL₃-PAA, Melittin-PLL, and KL₃-PLL with dimethylmaleic anhydride (DM) and 2-propionic-3-methylmaleic anhydride (CDM), general procedure: Peptide-polycation conjugates (10 mg/mL) in water were reacted with a ten-fold weight excess of dimethylmaleic anhydride and a ten-fold weight excess of potassium carbonate. Analysis of the amine content after 30 by addition of peptide solution to 0.4 mM TNBS and 100 mM borax revealed no detectable amounts of amine.

C. Polyvinyl(2-methyl-4-hydroxymethyl(succinic anhydride ester)-1,3-dioxolane: To a solution of polyvinyl(2-methyl-4-hydroxymethyl-1,3-dioxolane) (220 mg, 1.07 mmol) in dichloromethane (5 mL) was added succinic anhydride (161 mg, 1.6 mmol, Sigma Chemical Company), followed by diisopropylethyl amine (0.37 mL, 2.1 mmol, Aldrich Chemical Company) and the solution was heated at reflux. After 16 h, the solution was concentrated, dialyzed against H₂O (1×3 L, 3500 MWCO), and lyophilized to afford 250 mg (75%) of the ketal acid polyvinyl(2-methyl-4-hydroxymethyl(succinic anhydride ester)-1,3-dioxolane.

D. Ketal from Polyvinyl Alcohol and 4-Acetylbutyric Acid: Polyvinylalcohol (200 mg, 4.54 mmol, 30,000-60,000 MW, Aldrich Chemical Company) was taken up in dioxane (10 mL). 4-acetylbutyric acid (271 μL, 2.27 mmol, Aldrich Chemical Company) was added followed by p-toluenesulfonic acid monohydrate (86 mg, 0.45 mmol, Aldrich Chemical Company). After 16 h, TLC indicated the presence of ketone. The solution was concentrated under reduced pressure, and the residue dissolved in dimethylformamide (7 mL). The solution was heated to 60° C. for 16 h. After 16 h, TLC indicated the loss of ketone in the reaction mixture. Dialysis against H₂O (1×4 L, 3500 MWCO), followed by lyophilization resulted in 145 mg (32%) of the ketal. Ketone was not observed in the sample by TLC analysis, however, upon treatment with acid, the ketone was again detected.

E. Partial Esterification of Poly-Glutamic Acid with Di-(2-methyl-4-hydroxymethyl-1,3-dioxolane)-1,4-benzene (MC 196): To a solution of poly-L-glutamic acid (103 mg, 792 μmol, 32,000 MW, Sigma Chemical Company) in sodium phosphate buffer (30 mM) was added 1-(3-dimethylaminopropyl)-3-ethylcarbodiimide hydrochloride (129 mg, 673 μmol, Aldrich Chemical Company), followed by di-(2-methyl-4-hydroxymethyl-1,3-dioxolane)-1,4-benzene (25.0 mg, 80.5 μmol), and a catalytic amount of 4-dimethylaminopyridine. After 12 h, the reaction mixture was dialyzed against water (2×1 L, 12,000-14,000 MWCO) and lyophilized to afford 32 mg of poly-glutamic acid partially esterified with di-(2-methyl-4-hydroxymethyl-1,3-dioxolane)-1,4-benzene.

F. Aldehyde Derivatization of the Poly-Glutamic Acid Partially Esterified with Di-(2-methyl-4-hydroxymethyl-1,3-dioxolane)-1,4-benzene: To a solution of succinic semialdehyde (2.4 mg, 23 μmol, Aldrich Chemical Company) in water (100 μL) was added 1-(3-dimethyl-aminopropyl)-3-ethylcarbodiimide hydrochloride (4.7 mg, 2.4 μmol, Aldrich Chemical Company) followed by N-hydroxysuccinimide (2.8 mg, 24 μmol, Aldrich Chemical Company). The reaction was stirred at RT for 20 min. Formation of the N-hydroxysuccinic ester of succinic semialdehyde was confirmed by mass spectrometry.

Poly-glutamic acid partially esterified with di-(2-methyl-4-hydroxymethyl-1,3-dioxolane)-1,4-benzene (15.0 mg, 115 μmol) was taken up in water (100 μL) and added to the N-hydroxysuccinic ester of succinic semialdehyde, followed by a crystal of 4-dimethyl-aminopyridine. The reaction mixture was stirred overnight at RT. After 12 h the reaction mixture was dialyzed against water (2×1 L, 12,000-14,000 MWCO) and lyophilized to afford 3.0 mg. After dialysis the product tested positive for aldehyde content with 2,4-di-nitrophenylhydrazine.

G. polypropylacrylic acid: To a solution of diethylpropylmalonate (2 g, 10 mmol) in 50 mL ethanol was added potassium hydroxide (0.55 g, 1 eq) and the mixture was stirred at RT for 16 h. The ethanol was then removed by rotary evaporation. The reaction mixture was partitioned between 50 mL ethyl acetate and 50 mL of water. The aqueous solution was isolated, and acidified with hydrochloric acid. The solution was again partitioned between ethyl acetate and water. The ethyl acetate layer was isolated, dried with sodium sulfate, and concentrated to yield a clear oil. To this oil was added 20 mL of pyridine, paraformaldehyde (0.3 g, 10 mmol), and 1 mL piperidine. The mixture was refluxed at 130° C. until the evolution of gas was observed, ca. 2 h. The ester product was then dissolved into 100 mL ethyl ether, which was washed with 100 mL 1M hydrochloric acid, 100 mL water, and 100 mL saturated sodium bicarbonate. The ether layer was isolated, dried with magnesium sulfate, and concentrated by rotary evaporation to yield a yellow oil. The ester was then hydrolyzed by dissolving in 50 mL ethanol with addition of potassium hydroxide (0.55 gm, 10 mmol). After 16 h, the reaction mixture was acidified by the addition of hydrochloric acid. The propylacrylic acid was purified by vacuum distillation (0.9 g, 80% yield), boiling point of product is 60° C. at 1 torr. The propylacrylic acid was polymerized by addition of 1 mole percent of azobisisobutyonitrile and heating to 60° C. for 16 h. The polypropylacrylic acid was isolated by precipitation with ethyl ether.

H. 5,5′-Dithiobis(2-nitrobenzoic acid)-Poly-Glutamic acid (8 mer) Copolymer: H₂N-EEEEEEEE-NHCH₂CH₂NH₂ (5.0 mg, 0.0052 mmol, Genosis) was taken up in 0.1 mL HEPES (250 mM, pH 7.5). 5,5′-dithiobis[succinimidyl(2-nitrobenzoate)] (3.1 mg, 0.0052) was added with 0.2 mL DMSO and the mixture was stirred overnight at RT. After 16 h the solution was heated to 70° C. for 10 min, cooled to RT and diluted to 1.10 mL with DMSO.

### Example 5

### Synthesis of Polycations

A. L-cystine-1,4-bis(3-aminopropyl)piperazine copolymer: To a solution of N,N′-Bis(t-BOC)-L-cystine (85 mg, 0.15 mmol) in ethyl acetate (20 mL) was added N,N′-dicyclohexylcarbodiimide (108 mg, 0.5 mmol) and N-hydroxysuccinimide (60 mg, 0.5 mmol). After 2 h, the solution was filtered through a cotton plug and 1,4-bis(3-aminopropyl)piperazine (54 μL, 0.25 mmol) was added. The reaction was allowed to stir at RT for 16 h. The ethyl acetate was then removed by rotary evaporation and the resulting solid was dissolved in trifluoroacetic acid (9.5 mL), water (0.5 mL) and triisopropylsilane (0.5 mL). After 2 h, the trifluoroacetic acid was removed by rotary evaporation and the aqueous solution was dialyzed in a 15,000 MW cutoff tubing against water (2×2 1) for 24 h. The solution was then removed from dialysis tubing, filtered through 5 μM nylon syringe filter and then dried by lyophilization to yield 30 mg of polymer.

B. Adducts between peptides and polyamines: To a solution of poly-L-lysine (10 mg, 0.2 μmol) or polyallylamine (10 mg, 0.2 μmol) and peptides, such as KL₃ or melittin (2 μmol), was added 1-(3-dimethylaminopropyl)-3-ethylcarbodiimide hydrochloride (20 μmol). For the peptide KL₃, the reaction was performed in 2 mL water. For the peptide melittin, the reaction was performed in a solution of 1 mL water and 1 mL triflouroethanol. The reaction was allowed to proceed overnight before placement into a 12,000 molecular weight cutoff dialysis bag and dialysis against 4×2 liters over 48 h. The amount of coupled peptide was determined by the absorbance at 280 nm of a peptide tryptophan residue, using an extinction coefficient of 5690 cm⁻¹M⁻¹. The conjugate of melittin and poly-L-lysine was determined to have 4 molecules of melittin per molecule of poly-L-lysine and is referred to as Mel-PLL. The conjugate of KL₃ and poly-L-lysine was determined to have 10 molecules of KL₃ per molecule of poly-L-lysine and is referred to as KL₃-PLL. The conjugate melittin and polyallylamine was determined to have 4 molecules of melittin per molecule of polyallylamine and is referred to as Mel-PAA. The conjugate of KL₃ and polyallylamine was determined to have 10 molecules of KL₃ per molecule of polyallylamine and is referred to as KL₃-PAA.

C. Di-(2-methyl-4-hydroxymethyl(glyoxilic acid ester)-1,3-dioxolane)-1,4-benzene:1,4-Bis(3-aminopropyl)piperazine Copolymer (1:1) (MC228): To a solution of di-(2-methyl-4-hydroxymethyl(glyoxylic acid ester)-1,3-dioxolane) 1,4-benzene (100 mg, 0.273 mmol) in dimethylformamide was added 1,4-bis(3-aminopropyl)-piperazine (23 μL, 0.273 mmol, Aldrich Chemical Company) and the solution was heated to 80° C. After 16 h the solution was cooled to RT and precipitated with diethyl ether. The solution was decanted and the residue washed with diethyl ether (2×) and dried under vacuum to afford di-(2-methyl-4-hydroxymethyl(glyoxylic acid ester)-1,3-dioxolane) 1,4-benzene: 1,4-bis(3-aminopropyl)-piperazine copolymer (1:1). By similar methods the following polymers were constructed:

1. Di-(2-methyl-4-hydroxymethyl(succinic semialdehyde ester)-1,3-dioxolane)-1,4-benzene: 1,4-Bis(3-aminopropyl)piperazine Copolymer (1:1) (MC208).

2. Di-(2-methyl-4-hydroxymethyl(succinic semialdehyde ester)-1,3-dioxolane)-1,4-benzene: 1,4-Bis(3-aminopropyl)piperazine Copolymer (1:1) Reduced with NaCNBH₃ (MC301).

3. Di-(2-methyl-4-hydroxymethyl(succinic semialdehyde ester)-1,3-dioxolane)-1,4-benzene: 1,3-Diaminopropane Copolymer (1:1) (MC300).

4. Di-(2-methyl-4-hydroxymethyl(succinic semialdehyde ester)-1,3-dioxolane)-1,4-benzene: 3,3′-Diamino-N-methyldipropylamine Copolymer (1:1) (MC218).

5. Di-(2-methyl-4-hydroxymethyl(succinic semialdehyde ester)-1,3-dioxolane)-1,4-benzene: Tetraethylenepentamine Copolymer (1:1) (MC217).

6. Di-(2-methyl-4-hydroxymethyl(glyoxilic acid ester)-1,3-dioxolane)-1,4-benzene: 1,3-Diaminopropane Copolymer (1:1) (MC226).

7. Di-(2-methyl-4-hydroxymethyl(glyoxilic acid ester)-1,3-dioxolane)-1,4-benzene: 3,3′-Diamino-N-methyldipropylamine Copolymer (1:1) (MC227).

D. 1,4-Bis(3-aminopropyl)piperazine-Glutaric Dialdehyde Copolymer (MC140): 1,4-Bis(3-aminopropyl)piperazine (206 μL, 0.998 mmol, Aldrich Chemical Company) was taken up in 5.0 mL H₂O. Glutaric dialdehyde (206 μL, 0.998 mmol, Aldrich Chemical Company) was added and the solution was stirred at RT. After 30 min, an additional portion of H₂O was added (20 mL), and the mixture neutralized with 6 N HCl to pH 7, resulting in a red solution. Dialysis against H₂O (3×3 L, 12,000-14,000 MWCO) and lyophilization afforded 38 mg (14%) of the copolymer. By similar methods the following polymers were constructed:

1. Diacetylbenzene—1,3-Diaminopropane Copolymer (1:1) (MC321)

2. Diacetylbenzene—Diamino-N-methyldipropylamine Copolymer (1:1) (MC322).

3. Diacetylbenzene—1,4-Bis(3-aminopropyl)piperazine Copolymer (1:1) (MC229)

4. Diacetylbenzene—Tetraethylenepentamine Copolymer (1:1) (MC323).

5. Glutaric Dialdehyde—1,3-Diaminopropane Copolymer (1:1) (MC324)

6. Glutaric Dialdehyde—Diamino-N-methyldipropylamine Copolymer (1:1) (MC325).

7. Glutaric Dialdehyde—Tetraethylenepentamine Copolymer (1:1) (MC326).

8. 1,4-Cyclohexanone—1,3-Diaminopropane Copolymer (1:1) (MC330)

9. 1,4-Cyclohexanone—Diamino-N-methyldipropylamine Copolymer (1:1) (MC331).

10. 1,4-Cyclohexanone—1,4-Bis(3-aminopropyl)piperazine Copolymer (1:1) (MC312)

11. 1,4-Cyclohexanone—Tetraethylenepentamine Copolymer (1:1) (MC332).

12. 2,4-Pentanone—1,4-Bis(3-aminopropyl)piperazine Copolymer (1:1) (MC340)

13. 2,4-Pentanone—Tetraethylenepentamine Copolymer (1:1) (MC347).

14. 1,5-Hexafluoro-2,4-Pentanone—1,4-Bis(3-aminopropyl)piperazine Copolymer (1:1) (MC339)

15. 1,5-Hexafluoro-2,4-Pentanone—Tetraethylenepentamine Copolymer (1:1) (MC346).

E. Di-(2-methyl-4-aminomethyl-1,3-dioxolane)-1,4-benzene-Glutaric Dialdehyde Copolymer (MC352): To a solution of di-(2-methyl-4-aminomethyl-1,3-dioxolane)-1,4-benzene (23 mg, 75 μmol) in dimethylformamide (200 μL) was added glutaric dialdehyde (7.5 mg, 75 μmol, Aldrich Chemical Company). The reaction mixture was heated at 80° C. for 6 h under nitrogen. The solution was cooled to RT and used without further purification.

F. Di-(2-methy-4-hydroxymethyl(glycine ester)-1,3-dioxolane)-1,4,benzene—Glutaric Dialdehyde Copolymer (MC357): To a solution of di-(2-methy-4-hydroxymethyl(glycine ester)-1,3-dioxolane)-1,4,benzene (35 mg, 82 μmol) in dimethylformamide (250 μL) was added glutaric dialdehyde (8.2 mg, 82 μmol, Aldrich Chemical Company). The reaction mixture was heated at 80° C. for 12 b. The solution was cooled to RT and used without further purification.

G. Silyl Ether from Polyvinylalcohol and 3-Aminopropyltrimethoxysilane (MC221) pH-labile polyampholyte: To a solution of polyvinylalcohol (520 mg, 11.8 mmol (OH), 30,000-70,000 MW, Sigma Chemical Company) in dimethylformamide (4 mL) was added 3-aminopropyltrimethoxysilane (1.03 mL, 5.9 mmol, Aldrich Chemical Company) and the solution was stirred at RT. By similar methods the following polymers were constructed:

1. Silyl Ether from Poly-L-Arginine/-L-Serine(3:1) and 3-Aminopropyltrimethoxysilane (2:1) (MC358). Poly-L-Arginine/-L-Serine (20,000-50,000 MW, Sigma)

2. Silyl Ether from Poly-D,L-Serine and 3-Aminopropyltrimethoxysilane (3:1) (MC366). Poly-D,L-Serine (5,000-15,000 MW)

3. Silyl Ether from Poly-D,L-Serine and 3-Aminopropyltrimethoxysilane (2:1) (MC367). Poly-D,L-Serine (5,000-15,000 MW)

4. Silyl Ether from Poly-D,L-Serine and N-[3-(Triethoxysilyl)propyl]-4,5-dihydroimidizole (3:1) (MC369). Poly-D,L-Serine (5,000-15,000 MW)

5. Silyl Ether from Poly-D,L-Serine and N-Trimethoxysilylpropyl-N,N,N-trimethylammonium chloride (3:1) (MC370). Poly-D,L-Serine (5,000-15,000 MW)

6. Silazane from Poly-L-Lysine and 3-Aminopropyltrimethoxysilane (2:1) (MC360).

7. Poly(1,1-Dimethylsilazane) Tolemer (MC222).

H. 5,5′-Dithiobis(2-nitrobenzoic acid)-1,4-Bis(3-aminopropyl)piperazine Copolymer: 1,4-Bis(3-aminopropyl)piperazine (10 mL, 0.050 mmol, Aldrich Chemical Company) was taken up in 1.0 mL methanol and HCl (2 mL, 1 M in Et2O, Aldrich Chemical Company) was added. Et₂O was added and the resulting HCl salt was collected by filtration. The salt was taken up in 1 mL DMF and 5,5′-dithiobis[succinimidyl(2-nitrobenzoate)] (30 mg, 0.050 mmol) was added. The resulting solution was heated to 80° C. and diisopropylethylamine (35 mL, 0.20 mmol, Aldrich Chemical Company) was added by drops. After 16 h, the solution was cooled, diluted with 3 mL H₂O, and dialyzed in 12,000-14,000 MW cutoff tubing against water (2×2 L) for 24 h. The solution was then removed from dialysis tubing and dried by lyophilization to yield 23 mg (82%) of 5,5′-dithiobis(2-nitrobenzoic acid)-1,4-bis(3-aminopropyl)piperazine copolymer.

I. Cysteine-modified polycations: The N-hydoxysuccinimide (NHS) ester of N-Fmoc-S-tert-butylthio-L-cysteine was generated by reaction of protected amino acid with dicyclohexylcarbodiimide (DCC) and NHS in acetonitrile. After 16 h, the dicyclohexylurea is filtered off. The polycation is dissolved in methanol, ca 10 mg/ml, by the addition of 1 equivalent of diisopropylethylamine. To this polycation solution is added the NHS ester in acetonitrile. After 1 h, the modified polycation is precipitated out by the addition of ethyl ether. The modified polycation is then dissolved in piperidine and methanol (50/50). After 30 minutes, the cysteine-modified polycation is precipitated out by the addition of ethyl ether and then dissolved to 10 mg/ml in water. The pH of the solution is then reduced by the addition of concentrated hydrochloric acid to reduce the pH to 2.

J. Amine-containing enol ether copolymers (i.e. Poly(alkyl enolether-co-vinyloxy ethylamine) Polymers: 2-(vinyloxy)ethyl phthalimide (ImVE) was prepared by reacting 2-chloroethyl vinyl ether (25 g, 0.24 mol) with potassium phthalimide (25 g, 0.135 mol) in dimethyl foramide (75 mL) using tetra-n-butyl ammonium bromide as a phase transfer catalyst. This reaction mixture was stirred at 100° C. for 6 h then poured into 800 mL distilled water, and filtered and washed with a large amount of distilled water. The recovered yellowish crystals where then recrystallized twice from methanol to give white crystals, which were then dried for 48 h under reduced pressure. Polymerization was carried out in anhydrous methylene chloride at −78° C. under a blanket of dry nitrogen gas in oven-dried glassware. The reaction was initiated by adding borontrifluoride diethyl etherate to ImVE, and a mixture of enol ethers. The reaction was allowed to run for 3 h at −78° C., and then allowed to warm for ten minutes at RT, and then quenched with prechilled ammonia saturated methanol. The product was then evaporated to dryness under reduced pressure to give the product polymers. The polymer was then dissolved in a 1,4-dioxane(2)/methanol mixture and 10 equivalents (eq.) of hydrazine hydrate per mole of amine present. This solution was then refluxed for 2 h, cooled to RT, and the solvent was then removed under reduced pressure. This solution was then brought up in 0.5M HCl, and refluxed for 60 minutes. The cooled solution was then transferred to 3,000 MW dialysis tubing and dialyzed (4×5 L) for 48 h. This solution was then frozen and lyophilized. The following polymers were generated using this procedure (Table 1):

TABLE 1
Formulations for Amine-containing Enol Ether Copolymers

| | | equivalents added | | | |
|---|---|---|---|---|---|
| | | | octadecyl | ethyl enol | butyl enol |
| Polymer | BF₃EtOEt | ImVE | enol ether | ether | ether |
| DW#291 | 2% | 0.875 | 0.03 | 0.095 | — |
| DW#301 | 2% | 0.75 | 0.03 | — | 0.22 |
| DW#290 | 2% | 0.97 | 0.03 | — | — |

K. Poly(alkyl enolether-co-vinyloxy ethylamine) graft lactobionic acid polycation (DW#297): DW#290 (15,000 MW) was dissolved to 50 mg/mL in 100 mM MES (pH 6.5) buffer in a 15-ml polypropylene tube. To this solution was added 0.3 molar equivalent (relative to amine content of DW#290) lactobionic acid. N-(3-Dimethylaminopropyl)-N′ethylcarbodiimide (EDC) (0.33 equivalent) and N-hydroxysuccinimide (0.33 equivalent) were dissolved in 2 ml MES buffer and added immediately to the solution containing DW#290. The reaction tube was sealed and allowed to react at RT for 24 h. The reaction mixture was then removed from the tube and placed into dialysis tubing (3,500 MW cutoff), and dialyzed against 7×4 L water over a one week period. The polymer was then removed from the tubing and concentrated by lyophilization to 10 mg/mL.

### Example 6

### Demonstration of Lability of Labile Polyampholytes and Components

A. DM-poly-L-lysine: Dimethyl maleamic modified poly-L-lysine (10 mg/mL) was incubated in 10 mM sodium acetate buffer pH 5. At various times, aliquots (10 μg) were removed and added to 0.5 mL of 100 mM borax solution containing 0.4 mM trinitrobenzenesulfonate (TNBS). After 30 min, the absorbance of the solution at 420 nm was measured. To determine the concentration of amines at each time point, the extinction coefficient was determined for the product of TNBS and poly-L-lysine. Using this extinction coefficient we were able to calculate the amount of amines and maleamic groups at each time point. A plot of In (Aₜ/A₀) as a function of time was a straight line whose slope was the negative of the rate constant for the conversion of maleamic acid to amine and anhydride, where Aₜ is the concentration of maleamic acid at a time t and A₀ is the initial concentration of maleamic acid. For two separate experiments we calculated rate constants of 0.066 sec⁻¹ and 0.157 sec⁻¹ which correspond to half lives of roughly 10 and 4 minutes, respectively.

B. DM-KL₃: Dimethyl maleamic modified KL₃ (0.1 mg/mL) was incubated in 40 mM sodium acetate buffer pH 5 and 1 mM cetyltrimetylammonium bromide. At various times, 10 μg aliquots were removed and added to 0.05 mL 1 M borax solution containing 4 mM TNBS. After 30 min, the absorbance of the solution at 420 nm was measured. To determine the concentration of amines at each time point, the extinction coefficient was determine for the product of TNBS and KL₃. Using this extinction coefficient we were able to calculate the amount of amines and maleamic groups at each time point. A plot of In (Aₜ/A₀) as a function of time was a straight line whose slope is the negative of the rate constant for the conversion of maleamic acid to amine and anhydride, where Aₜ is the concentration of maleamic acid at a time t and A₀ is the initial concentration of maleamic acid. We calculated a rate constant of 0.087 sec⁻¹ that corresponds to a half-life of roughly 8 minutes.

C. Membrane active compounds Melittin and KL₃ and their dimethylmaleamic acid derivatives: The membrane-disruptive activity of the peptide melittin and subsequent blocking of activity by anionic polymers was measured using a red blood cell (RBC) hemolysis assay. RBCs were harvested by centrifuging whole blood for 4 min. They were washed three times with 100 mM dibasic sodium phosphate at the desired pH, and resuspended in the same buffer to yield the initial volume. They were diluted 10 times in the same buffer, and 200 μL of this suspension was used for each tube. This yields 108 RBCs per tube. Each tube contained 800 μL of buffer, 200 μL of the RBC suspension, and the peptide with or without polymer. Each sample was then repeated to verify reproducibility. The tubes were incubated for 30 minutes in a 37° C. water bath. They were spun for 5 min at full speed in the microcentrifuge. Lysis was determined by measuring the absorbance of the supernatant at 541 nm, reflecting the amount of hemoglobin that had been released into the supernatant. Percent hemolysis was calculated assuming 100% lysis to be measured by the hemoglobin released by the red blood cells in water; controls of RBCs in buffer with no peptide were also run. The results, shown in Table 2, indicate that dimethylmaleamic modification of the peptides KL3 and Melittin inhibits their activity in a pH dependent manner. Activity of these membrane active compounds is regenerated at acidic pH.

TABLE 2
pH-dependent activation of dimethylmaleamic-
modified membrane active polycations

Percent
Hemolysis

| Peptide | pH 5.4 | pH 7.5 |
|---|---|---|
| KL₃ | 83 | 62 |
| DM-KL₃ | 37 | 4.3 |
| Succinyl-KL₃ | 2.0 | 1.3 |
| Melittin | 85 | 92 |
| DM-Melittin | 100 | 1.0 |
| Succinyl-Melittin | 5.0 | 2.0 |

### Example 7

Inhibition of gene expression in lung following delivery of siRNA using siRNA/brPEI-pAA polyampholytes: In this example we show that polyampholyte complexes can be used for in vivo cellular delivery of siRNA. The delivered siRNA inhibits gene expression in a sequence-specific manner. To demonstrate functional delivery of siRNA to lung, mice were first transfected with two distinct luciferase genes encoding either firefly and renilla luciferase using recharged plasmid DNA/lPEI/polypropylacrylic acid complexes.

Plasmid DNA complexes were prepared by combining 49.5 μg pMIR116 (firefly luciferase plasmid vector) and 0.5 μg pMIR122 (renilla luciferase plasmid vector) with 200 μg linear-PEI in 5 mM HEPES pH 7.5/290 mM glucose. 50 μg polyacrylic acid was then added to recharge the complexes. The complexes, in a total volume of 250 μl, were then injected into the tail vain of each mouse. Two hours after injection of recharged DNA complexes, mice were injected via tail vain with 250 μl injection solution containing siRNA/polyampholytes complexes made with 50 μg firefly luciferase specific siRNA-luc+.

siRNA: Single-stranded, gene-specific sense and antisense RNA oligomers with overhanging 3′ deoxynucleotides were prepared and purified by PAGE (Dharmacon, LaFayette, Colo.). The two complementary oligonucleotides, 40 μM each, were annealed in 250 μl 100 mM NaCl/50 mM Tris-HCl, pH 8.0 buffer by heating to 94° C. for 2 minutes, cooling to 90° C. for 1 minute, then cooling to 20° C. at a rate of 1° C. per minute. The resulting siRNA was stored at −20° C. prior to use. The sense oligonucleotide, with identity to the luc+gene in pGL-3-control, had the sequence: 5′-rCrUrUrArCrGrCrUrGrArGrUrArCrUrUrC-rGrATT-3′ (SEQ ID 2), corresponding to positions 155-173 of the luc+reading frame. The antisense oligonucleotide, with identity to the luc+gene in pGL-3-control, had the sequence: 5′-rUrCrGrArArGrUrArCrUrCrArGrCrGrUrArArGTT-3′ (SEQ ID 3) corresponding to positions 173-155 of the luc+reading frame in the antisense direction. The letter “r” preceding a nucleotide indicates that the nucleotide is a ribonucleotide. The annealed oligonucleotides containing luc+coding sequence are referred to as siRNA-luc+. Polyampholyte: Branched PEI-pAA polyampholyte was prepared as described in example 1A above.

Injection solution contained siRNA complexed with varying amounts of polyampholytes. Complexes were prepared using 50 μg siRNA and the indicated amount of brPEI-poly(aspartic acid) polyampholyte. Polyampholyte was mixed with siRNA in 5 mM HEPES pH 7.5/290 mM glucose, 250 μl total volume, and injected within 1 h of complex preparation. Controls included siRNA/brPEI complexes and siRNA/brPEI/pAsp complexes. 24 h after siRNA complex injection, lung tissue was harvested and assayed for luciferase activity using the Promega Dual Luciferase Kit (Promega) and a Lumat LB 9507 luminometer (EG&G Berthold, Bad-Wildbad, Germany). The amount of luciferase expression was recorded in relative light units. Numbers were adjusted for control renilla luciferase expression and are expressed as the percentage of firefly luciferase expression in mice that did not receive injections containing siRNA.

Conclusions: Complexes containing siRNA/brPEI were toxic to the animals and provided no inhibition of firefly luciferase activity (4 of 5 animal killed). SiRNA/brPEI complexes recharged with pAsp polymer were less toxic that siRNA/brPEI complexes, but did not result in siRNA mediated inhibition of luciferase activity (10-20% inhibition of luciferase expression). However, when siRNA-containing complexes were made using brPEI-pAsp polyampholytes, PEI toxicity was reduced and siRNA was functionally delivered to lung cells. Polyampholyte-mediated delivery of siRNA resulted in the gene-specific inhibition of firefly luciferase expression by 60% (FIG. X).

### Example 8

Delivery of siRNA to cells in vitro using polyampholytes The polyampholyte brPEI-pAsp (2:1 w/w) was synthesized as in example 1A. COS7 cells were initially transfected with two distinct luciferase genes encoding either firefly and renilla luciferase genes (pMIR116 and pMIR122, respectively) using TransITLT1 according to the manufacturer's recommendations. Two hours after plasmid transfection, siRNA/polyampholyte complexes were added to cells. SiRNA/brPEI-pAsp complexes were prepared in 10 mM HEPES, 150 mM NaCl, pH 7.5 (HBS) immediately prior to transfections. The transfections were done in Opti-MEM supplemented with 10% fetal bovine serum. The concentration of siRNA was 40 nM. Luciferase activity was measured 24 h post-transfection. SiRNA delivery was measured by the ratio of firefly to renilla luciferase activity in the presence or absence of firefly specific siRNA. The data are shown in FIG. and show that brPEI-pAsp polyampholyte complexes are effective in delivering siRNA to cells in vitro.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

### REFERENCES

Asayama S, Nogawa M, Takei Y, Akaike T, Maruyama A. Synthesis of Novel Polyampholyte Comb-Type Copolymers Consisting of a Poly(L-lysine) Backbone and Hyaluronic Acid Side Chains for a DNA Carrier. Bioconjugate Chem 1998; 9(4):476-481.

Blattler W A, Kuenzi B S, Lambert J M, Senter P D. New heterobifunctional protein crosslinking reagent that forms an acid-labile link. Biochemistry 1985; 24(6): 1517-1524.

Bloomfield V A. DNA condensation. Curr Opin Struct Biol 1996; 6(3):334-341.

Dash P R, Read M L, Barrett L B, Wolfert M A, Seymour L W. Factors Affecting Blood Clearance and In Vivo Distribution of Polyelectrolyte Complexes for Gene Delivery. Gene Therapy 1999; 6:643-650.

Elbashir S M, Harborth J, Lendeckel W, Yalcin A, Weber K, Tuschl T. Duplexes of 21-nucleotide RNAs mediate RNA interference in cultured mammalian cells. Nature 2001; 411(6836):494-498.

Gill S C, von Hippel P H. Calculation of protein extinction coefficients from amino acid sequence data. Analytical Biochemistry 1989; 182(2):319-326.

Meyer O, Papahadjopoulos D, Leroux J C. Copolymers of N-isopropylacrylamide can trigger pH sensitivity to stable liposomes. FEBS Lett. 1998; 421(1):61-64.

Murthy N, Robichaud J R, Tirrell D A, Stayton P S, Hoffman A S. The design and synthesis of polymers for eukaryotic membrane disruption. J Control Release. 1999; 61(1-2):137-143.

Netz R R, Joanny J F. Complexation Behavior of Polyampholytes and Charged Objects. Macromolecules. 1998; 31(15):5123-5141.

Ogris M, Brunner S, Schuller S, Kircheis R, Wagner E. PEGylated DNA/Transferrin-PEI Complexes: Reduced Interaction with Blood Components, Extended Circulation in Blood and Potential for Systemic Gene Delivery. Gene Therapy 1999; 6:595-605.

Plank C, Mechtler K, Szoka F C Jr, Wagner E. Activation of the Complement System by Synthetic DNA Complexes: A Potential Barrier for Intravenous Gene Delivery. Human Gene Therapy 1996; 7:1437-1446.

Ross P C, Hui S W. Lipoplex size is a major determinant of in vitro lipofection efficiency. Gene Ther. 1999; 6(4):651-659.

Sharp P A. RNA interference—2001. Genes Dev 2001; 15:485-490.

Stevens M P. Polymer Chemistry: An Introduction. New York Oxford University Press 1990.

Trubetskoy V S, Loomis A, Hagstrom J E, Budker V G, Wolff J A. Layer-by-layer deposition of oppositely charged polyelectrolytes on the surface of condensed DNA particles. Nucleic Acids Res. 1999; 27(15):3090-3095.

Tuschl T, Zamore, P D, Lehmann R, Bartel D P, Sharp P A. Targeted mRNA degradation by double-stranded RNA in vitro. Genes Dev 1999; 13, 3191-3197.

Vitiello L, Bockhold K, Joshi P B, Worton R G. Transfection of cultured myoblasts in high serum concentration with DODAC:DOPE liposomes. Gene Ther. 1998; 5(10):1306-1313.

Wolfert M A, Schacht E H, Toncheva V, Ulbrich K, Nazarova O, Seymour L W. Characterization of Vectors for Gene Therapy Formed by Self-Assembly of DNA with Synthetic Block Co-Polymers. Human Gene Therapy 1996; 7:2123-2133.

Xu, Yuhong, Et al., Mechanism of DNA Release from Cationic Liposome/DNA Complexes Used in Cell Transfection. Biochemistry 1996; 35:5616-5623.

We claim:

1. A process for enhancing delivery of siRNA to a cell, comprising:
a) forming a complex of polyampholyte and siRNA; and,
b) delivering the complex into a cell.

2. The process of claim 1 wherein the polyampholyte comprises a polycation selected from group consisting of PLL and PEI.

3. The process of claim 1 wherein the polyampholyte comprises a polyanion.

4. The process of claim 3 wherein the polyanion comprises a molecule selected from the group consisting of succinylated PLL, succinylated PEI, polyglutamic acid, polyaspartic acid, polyacrylic acid, polymethacrylic acid, dextran sulfate, heparin, hyaluronic acid, DNA, RNA, and negatively charged proteins.

5. The process of claim 3 wherein the polyanion comprises a molecule selected from the group consisting of pegylated derivatives, pegylated derivatives carrying specific ligands, block copolymers, graft copolymers and hydrophilic polymers.

6. The process of claim 1 wherein the polyampholyte is delivered to a cell in vivo.

7. A complex for delivering siRNA to a cell, comprising:
a) siRNA; and,
b) a polyampholyte wherein the siRNA and the polyampholyte are bound in complex.

8. The complex of claim 7 wherein the polyampholyte comprises a polycation.

9. The complex of claim 8 wherein the polycation is selected from group consisting of PLL, PEI, histones or cationic lipids.

10. The complex of claim 7 wherein the polyampholyte comprises a polyanion.

11. The complex of claim 10 wherein the polyanion comprises a molecule selected from the group consisting of succinylated PLL, succinylated PEI, polyglutamic acid, polyaspartic acid, polyacrylic acid, polymethacrylic acid, dextran sulfate, heparin, hyaluronic acid, DNA, RNA, and negatively charged proteins.

12. The complex of claim 11 wherein the polyanion comprises a molecule selected from the group consisting of pegylated derivatives, pegylated derivatives carrying specific ligands, block copolymers, graft copolymers and hydrophilic polymers.

13. A process for extravasation of a complex, comprising:
a) forming a complex of polyampholyte and siRNA; and,
b) inserting the complex into a vessel;
c) delivering the complex to an extravascular space.

14. The process of claim 13 wherein the polyampholyte comprises a polycation selected from group consisting of PLL, PEI, histones or cationic lipids.

15. The process of claim 13 wherein the polyampholyte comprises a polyanion selected from the group consisting of succinylated PLL, succinylated PEI, polyglutamic acid, polyaspartic acid, polyacrylic acid, polymethacrylic acid, dextran sulfate, heparin, hyaluronic acid, DNA, RNA, and negatively charged proteins.

16. The process of claim 15 wherein the negatively charged polyion comprises a molecule selected from the group consisting of pegylated derivatives, pegylated derivatives carrying specific ligands, block copolymers, graft copolymers and hydrophilic polymers.

17. The process of claim 13 wherein the complex is delivered to an extravascular cell.

18. The process of claim 13 wherein the siRNA is delivered to an extravascular cell in vivo.

19. The process of claim 18 wherein the siRNA inhibits gene expression.


SEQUENCE LISTING (3 sequences)

SEQ ID NO: 1 - protein, 26 aa, Apis mellifera
GIGAVLKVLTTGLPALISWIKRKRQQ

SEQ ID NO: 2 - DNA, 21 bp, Photinus pyralis
cuuacgcugaguacuucgatt

SEQ ID NO: 3 - DNA, 21 bp, Photinus pyralis
ucgaaguacucagcguaagtt